(19) United States
(12) Patent Application Publication
(10) Pub. No.: US 2024/0301477 A1
(43) Pub. Date: Sep. 12, 2024

(54) A BIOSENSOR DEVICE, SYSTEM AND KIT FOR DETECTING INFECTION AND ANTIMICROBIAL RESISTANCE

(71) Applicant: Ramja Genosensor Private Limited, Delhi (IN)

(72) Inventor: Pooja Goswami, Delhi (IN)

(73) Assignee: Ramja Genosensor Private Limited, Delhi (IN)

(21) Appl. No.: 17/782,352

(22) PCT Filed: Dec. 3, 2020
(86) PCT No.: PCT/IB2020/061432

§ 371 Date: Jun. 3, 2022

(30) Foreign Application Priority Data
Dec. 3, 2019 (IN) 201911049726
Dec. 3, 2019 (IN) 201911049728
Dec. 3, 2019 (IN) 201911049729

(51) Int. Cl. C12Q 1/6825; C12Q 1/04; C12Q 1/6834; C12Q 1/689; G01N 33/533

(52) U.S. Cl. CPC C12Q 1/6825; C12Q 1/04; C12Q 1/6834; C12Q 1/689; G01N 33/533

(57) ABSTRACT

The present disclosure relates to a bio-sensor device, system, acrylamide cassette and kit for identifying a microorganism in a sample. The device can include a housing and a sensor cabinet comprising a receptacle portion for receiving a sensor comprising one or more working electrodes capable of oxidation-reduction process, wherein the sensor is configured to receive the biological sample and provide a surface for immobilization of a set of hybridization probes that form a three-dimensional complex upon independent hybridization with a target nucleic acid sequence of a gene corresponding to the microorganism, wherein the sensor upon being placed in the sensor cabinet enables detection of the three-dimensional complex by the oxidation-reduction process that generates an electrical signal measurable by the device that facilitates the identification of the MDR, XDR and PDR microorganism in the biological sample in DNA-based detection of any or a combination of an infection and an antimicrobial resistance caused by the microorganism.

### PRIORITY

The present application claims the benefit of the Indian provisional Application No. 201911049728 filed on Dec. 3, 2019, Indian provisional Application No. 201911049729 filed on Dec. 3, 2019 and Indian provisional Application No. 201911049726 filed on Dec. 3, 2019, the entire disclosures of which are relied on for all purposes and are incorporated into this application by reference.

### TECHNICAL FIELD

The present disclosure pertains to a paper based biosensor device. In particular the present disclosure relates to a biosensor device and a kit for identifying microorganisms in a given sample for DNA-based detection of infection and antimicrobial resistance in a shorter time duration within 2 hours.

### BACKGROUND

Infections are one of the major causes of mortality all over the world, especially in third world countries. Intensive care units (ICUs) are an important source of infection in a country like India. Mortality rate due to infection is high and is still increasing due to misuse of antibiotics. To prevent infection, there are basically seven classes of antibiotics which are available and all of them are essentially based upon five types of mechanism of action that is on a cell wall, cell membrane, nucleic acid, ribosomal unit and folate synthesis. In 2010, India was the world's largest consumer of antibiotics for human health. Due to over usage of these antibiotics, bacteria developed drug resistance using the same mechanism, on which drugs were discovered including an extended spectrum beta-lactamase (ESBL) resistance, carbapenems resistance and other types of resistance.

Being such a serious challenge, infection detection is still dependent on techniques such as bacterial culture, polymerase chain reaction (PCR), sequencing, matrix-assisted laser desorption/ionization (MALDI), which are very time consuming, lengthy, cumbersome, labour intensive and extremely costly, requiring highly specialized set up with very expensive instruments. Yet, such techniques and instruments do not provide results in shorter duration, wherein these tests generate results after 3-7 days. Moreover, the existing equipment are not easily movable for setting-up detection system at the site of the patient. Hence, there does exists an urgent unmet need for a portable system for identification of microorganisms to detect infection in less time with higher sensitivity and specificity.

Further, prophylactic treatment to this infection leads to multi antibiotic resistance and increase the mortality rate. It is known that An hour delay in antibiotic treatment to ICU patient (sepsis with septic shock) may cause 7-10% increase in mortality. As already known, β lactam or penicillin antimicrobial agent are the most common treatment for bacterial infection and continue to become most common cause of antibiotic resistance among the gram-negative bacterial infection. The reason behind this antibiotic resistance is extended-spectrum-lactamases (ESBLs) producing Enterobacteriaceae species due to persistence exposure of β lactam antibiotics. Hence, due to complications related to infection and lack of immediate culture results, physician are compelled to start prophylactic antibiotic medication to treat infection, especially in patients having acute leukaemia (haematological malignancies and) and other such immunocompromised diseases, in a set regimen that is the on the first day, antibiotics against gram negative bacteria, third day, antibiotics against gram positive followed by antifungal treatment on the fifth day without waiting for culture report. This may lead to developing antibiotic resistance due to prolonged exposure to antibiotics and thus may increase fatality.

In view of the above, it is evident that there is an urgent requirement of providing a means for detection of infection detection and antibiotic resistance which can overcome one or more shortcomings of the existing modalities, in terms of giving qualitative and quantitative test results, in simple, cost-effective and instantaneous manner.

### OBJECTS OF THE PRESENT DISCLOSURE

Some of the objects of the present disclosure, which at least one embodiment herein satisfies are as listed herein below.

It is an object of the present disclosure to provide a biosensor device for identifying microorganism(s) in a biological sample.

It is an object of the present disclosure to provide a biosensor device for identifying microorganism(s) in a given sample for detecting infection and antimicrobial resistance.

It is another object of the present disclosure to provide a portable device for identifying microorganism(s) in a given sample in DNA/plasmid DNA-based detection of an infection and antimicrobial resistance simultaneously in a shorter duration (less than 2 hour).

It is an object of the present disclosure to provide a biosensor device with high sensitivity to presence of microorganism that cause infection and/or antibiotic resistant bacterial cells.

It is an object of the present disclosure to provide a biosensor device capable of identifying infection causing and/or antibiotic resistant bacterial cells in a given sample in qualitative and quantitative manner.

It is an object of the present disclosure to provide a biosensor device for identifying infection causing and/or antibiotic resistant bacterial cells in a given sample with specificity for DNA based detection of infection, and their antibiotic resistance.

It is an object of the present disclosure to provide DNA biosensor device with a simpler set-up for identifying infection causing and/or antibiotic resistant bacterial cells in a given sample in cost effective manner.

It is an object of the present disclosure to provide a biosensor device that can be used as a point of care device for fast detection of infection or antibiotic resistance to enable physician to arrive at an immediate and appropriate antibiotic treatment regimen.

### SUMMARY

In an aspect, the present disclosure relates to a portable bio-sensor device for identifying a microorganism in a biological sample for DNA-based detection. The device can include a housing for enclosing one or more components of the device; and a sensor cabinet provided within the housing, wherein the sensor cabinet comprises a receptacle portion. The device can include a potentiometer arrangement that enables measurement of oxidation-reduction process, wherein the potentiometer may be configured to apply a pre-defined potential, measure a current output and estimate a number of cells corresponding to the microorganism present within the sample based on the current output, for DNA-based detection. In an embodiment, the housing can be cuboid-shaped and the device can include a display screen to indicate the generation data. In an embodiment, the device can include a thermal printer for printing the generated data.

In another aspect, the present disclosure provides a sensor for identifying a microorganism in a biological sample for DNA-based detection, wherein the sensor can include a working electrode that can interact with at least one capture probe and at least one detector probe, wherein the at least one capture probe comprises a first oligonucleotide sequence and the at least one detector probe can include a second oligonucleotide sequence, wherein each of the first oligonucleotide sequence and the second oligonucleotide sequence are single-stranded oligonucleotides that are complementary to a nucleic acid sequence hosted by the micro-organism. In an embodiment, the capture probe and the detector probe upon coming in contact with the nucleotide sequence hosted by the micro-organism affords formation of a three-dimensional complex through independent hybridization. In an embodiment, the one or more working electrodes may be carbon-based electrodes printed on a cellulosic substrate. In an exemplary embodiment, the cellulosic substrate may be paper.

In an embodiment, the formation of the three-dimensional complex can be detected by the one or more working electrodes by an oxidation-reduction process that enables generation of an electrical signal for identifying the strain of the microorganism in the sample in the detection of at least one of the infection and the antimicrobial resistance. In an embodiment, the sensor can include a reference electrode and a counter electrode coupled to the working electrode for measurement of the electrical signal in form of dataset including measurement of voltage and current associated with the oxidation-reduction process.

In an embodiment, the capture probe may be immobilized on the working electrode by a carrier molecule including protein, wherein the protein is selected from streptavidin and avidin, wherein the at least one capture probe defines a single-stranded oligonucleotide tagged with a conjugating agent at any of 5′ end or at 3′ end of the oligonucleotide, the conjugating agent being capable of being conjugated with the protein, wherein the conjugating agent is biotin, wherein the at least one detector probe defines a single-stranded oligonucleotide tagged with a conjugating agent at any of 5′ end or at 3′ end of the oligonucleotide, the conjugating agent being capable of being conjugated with the protein, wherein the conjugating agent is fluorescein.

In an embodiment, the wherein the detection of the formation of the three-dimensional complex may be done by using one or more reagents selected from any or a combination of anti-fluorescein monoclonal Fab fragment, horseradish peroxidase, a buffer, reagents for cell lysis and detection reagents, wherein the formation of the three-dimensional complex is carried out inside an acrylamide cassette, wherein the sensor is placed inside the cassette to enable hybridization between the probes and the target nucleic acid sequence on the surface of the sensor.

In another aspect, the present disclosure provides a system for identifying a microorganism in a biological sample for DNA-based detection. The system includes a device and a sensor as described herein above.

In an embodiment, the system can detect the microorganism that is a microbial strain selected from wild type strain, a pathogenic strain, an antibiotic resistant bacterial strain, a multidrug resistant bacterial strain, an extreme drug-resistant strain, and pan drug-resistant strain, wherein the detection of the sample for identifying the microorganism is done in a time duration in the range of 1 minute to 120 minutes, and wherein the biological fluid is selected from blood, urine and other biological fluids of a body. In an embodiment, the system enables detection of antimicrobial resistance caused by a gram negative bacterium selected from a strain of E. coli, Klebsiella pneumoniae and Pseudomonas aeruginosa.

In another aspect, the present disclosure relates to a set of hybridization probes for hybridizing with a nucleic acid sequence of a microorganism in a sample for DNA-based detection, wherein the set of hybridization probes comprise a capture probe having a first oligonucleotide sequence and a detector probe comprising a second oligonucleotide sequence,

In an embodiment, the first oligonucleotide sequence is selected from SEQ ID No. 1. SEQ ID No. 3, SEQ ID No. 5, SEQ ID No. 7, SEQ ID No. 9 and SEQ ID No. 11.

In an embodiment, the second oligonucleotide sequence is selected from SEQ ID No. 2, SEQ ID No. 4, SEQ ID No. 6, SEQ ID No. 8 SEQ ID No. 10 SEQ ID No. 12 and SEQ ID No. 14.

In an embodiment, the set of hybridization probes can be capable of individually hybridizing to any of the target nucleic acid sequence of the gene selected from CTX-M, KPC gene, and NDM-1 gene of Klebsiella pneumoniae strains to identify the strain resistant to antibiotics selected from carbapenem, amino glycosidase and colistin in detection of any or a combination of multi-drug resistance, extensively drug-resistance and pan drug-resistance, and wherein the set of hybridization probes are capable of hybridizing to the target nucleic acid sequence of 16s RNA gene of Klebsiella pneumoniaeto identify a virulent strain and to detect the infection.

In an embodiment, the set of hybridization probes may be capable of individually hybridizing to 16S rRNA of E. coli, Klebsiella pneumonia and Pseudomonas aeruginosa and the probes are used as marker to identify the pathogenic strain and discriminating from the pathogenic and drug-resistant strains of Pseudomonas aeruginosa.

In an embodiment, the set of hybridization probes may be capable of individually hybridizing to any of the target nucleic acid sequence of the gene selected from CTX-M, NDM-1, and KPC in extended spectrum beta-lactamase (ESBL) producing E. coli resistant to antibiotics selected from cephalosporins belonging to one to four classes, fluoroquinolones, and monobactam in detection of any or a combination of multi-drug resistance.

In an embodiment, the set of hybridization probes are capable of individually hybridizing the target nucleic acid sequence of the gene selected from CTX-M. KPC, and NDM-1 of Pseudomonas aeruginosa, to identify the strain resistant to antibiotics selected from carbapenem, amino glycosidase and colistin in detection of any or a combination of multi-drug resistance, extensively drug-resistance and pan drug-resistance, In another aspect, the present disclosure provides a kit for identifying a microorganism in a sample for DNA-based detection. The kit can include a cocktail mixture, a set of hybridization probes and one or more reagents, wherein the cocktail mixture is capable of lysing the gram negative bacterium and releasing nucleic acid in a biological sample in a time period in the range of 20 to 40 minutes, wherein the cocktail mixture includes a combination of 1M sodium hydroxide solution, 10% Tween, 20 mM Tris (hydroxymethyl) aminomethane hydrochloride (Tris HCl), 1 mM EDTA, Lysozyme (10 mg/ml) in 10 mM tris HCL in a volume ratio of 1:1:1:0.5:0.25, wherein the one or more reagents can be selected from any or a combination of streptavidin, biotin, anti-fluorescein monoclonal Fab fragment, horseradish peroxidase, a buffer, reagents for cell lysis and detection reagents. The set of hybridization probes can include capture probes and detector probes complementary to and capable of hybridizing to a target nucleic acid sequences of a gene for identification of infection causing and drug resistant strain(s) of the microorganism.

The device, system, cassette and kit of the present disclosure provide a convenient, rapid and cost-effective way of identifying a microorganism in a sample for DNA-based detection of any or a combination of the infection and the antimicrobial resistance.

Various objects, features, aspects and advantages of the inventive subject matter will become more apparent from the following detailed description of preferred embodiments.

### BRIEF DESCRIPTION OF DRAWINGS

The accompanying drawings are included to provide a further understanding of the present disclosure and are incorporated in and constitute a part of this specification. The drawings illustrate exemplary embodiments of the present disclosure and, together with the description, serve to explain the principles of the present disclosure.

The diagrams are for illustration only, which thus is not a limitation of the present disclosure, and wherein:

FIG. 1 illustrates an exemplary biosensor device 100 in accordance with an embodiment of the present disclosure.

FIG. 2 illustrates an exemplary cassette of a kit, in accordance with an embodiment of the present disclosure.

FIG. 3 illustrates a voltammetry data using biosensor device, in accordance with an embodiment of the present disclosure.

FIGS. 4A-4C illustrate cyclic voltammetry data in detection of an infection due to Pseudomonas aeruginosa, E. coli and Klebsiella pneumoniae respectively, in accordance with an embodiment of the present disclosure.

FIGS. 5A-5C illustrate cyclic voltammetry data in detection of antimicrobial resistance due to Pseudomonas aeruginosa, E. coli and Klebsiella pneumoniae respectively, in accordance with an embodiment of the present disclosure.

FIGS. 6A-6C illustrate cyclic voltammetry data in universal probe based detection of Pseudomonas aeruginosa, E. coli and Klebsiella pneumoniae respectively, in accordance with an embodiment of the present disclosure.

FIG. 7 illustrates cyclic voltammetry data for E. coli infection sensor of varying dilution developed by using Dh-5a as wild strain and MTCC 4296, in accordance with an embodiment of the present disclosure.

FIGS. 8A-8D illustrate cyclic voltammetry data that show oxidation reduction cycles of various E. coli probes, in accordance with embodiments of the present disclosure.

FIG. 9A illustrates cyclic voltammetry data that show oxidation reduction cycles depicting pattern of positivity of 16s RNA probes in term of reduction current, in accordance with an embodiment of the present disclosure.

FIG. 9B illustrates cyclic voltammetry data that show oxidation reduction cycles of an infection biosensor having specificity of infection probes corresponding to rfb gene and 16 sRNA, in accordance with an embodiment of the present disclosure.

FIG. 10 illustrates cyclic voltammetry data showing positivity for species specific drug resistant probe CTX-M1 in E. coli NCIM-2571, in accordance with an embodiment of the present disclosure.

### DETAILED DESCRIPTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Not withstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms are used herein. To the extent a term used in a claim is not defined below, it should be given the broadest definition persons in the pertinent art have given that term as reflected in printed publications and issued patents at the time of filing.

The oxidation-reduction potential means the standard potential of an atom or ion that undergoes oxidation at the anode or reduction at the cathode in an electrochemical cell as compared to the redox potential of a standard carbon-based printed electrodes

In an aspect, the present disclosure relates to a portable bio-sensor device for identifying a microorganism in a biological sample for DNA-based detection. The device can be designed and customized for detecting a wide range of microorganisms causing the infection (gram positive, TB, sepsis, fungal, viral, hospital acquired). In an embodiment, the device can detect a microbial strain selected from wild type strain, a pathogenic strain, an antibiotic resistant bacterial strain, a multidrug resistant bacterial strain, an extreme-drug-resistant strain, and pan drug-resistant strain. The device of the present disclosure can be an effective alternative for most of the existing modalities in term of time, experimental-expenditure, human labor and cost for infection detection and antibiotic resistance to obtain the results in a qualitative and quantitative manner.

In an embodiment, the device can include a housing for enclosing one or more components of the device; and a sensor cabinet provided within the housing, wherein the sensor cabinet comprises a receptacle portion. The device can include a potentiometer arrangement that enables measurement of oxidation-reduction process, wherein the potentiometer may be configured to apply a pre-defined potential, measure a current output and estimate an amount of cells corresponding to the microorganism present within the sample based on the current output for DNA-based detection.

As per an embodiment of the present disclosure, the bio-sensor device can include a housing for enclosing one or more components of the device and a sensor cabinet. The sensor cabinet can include a receptacle portion for receiving a sensor. A sensor may be a chip having one or more working electrodes capable of oxidation-reduction process. In an embodiment, the sensor can be configured to receive the biological sample and provide a surface for immobilization of a set of hybridization probes that form a three-dimensional complex upon independent hybridization with a target nucleic acid sequence of a gene corresponding to the microorganism in the biological sample to be identified, wherein the sensor upon being placed in the sensor cabinet may enable detection of the three-dimensional complex by the oxidation-reduction process that enables generation of an electrical signal measurable by the device that may facilitate the identification of the microorganism in the biological sample in DNA-based detection of any or a combination of an infection and an antimicrobial resistance caused by the microorganism.

In an embodiment, the set of hybridization probes can include a capture probe and a detector probe. The capture probe can include a first oligonucleotide sequence that may be a single-stranded oligonucleotide. The first oligonucleotide sequence may be tagged with a conjugating agent so that the capture probe can be localised on the one or more electrodes. The conjugating agent may be any agent capable of conjugating with a protein. In an exemplary embodiment, the conjugating agent can be biotin that may be tagged at 5-′ or 3′-end of the first oligonucleotide.

In an embodiment, the detector probe can include a second oligonucleotide sequence that may be a single-stranded oligonucleotide. The second oligonucleotide sequence may be tagged with at least one fluorescent compound or marker. In an exemplary embodiment, the fluorescent compound can be fluorescein that may be tagged at 5-′ or 3′-end of the second oligonucleotide.

In an embodiment, the one or more working electrodes may be functionalized by coating with at least one protein compound. The functionalized protein on the electrodes may provide a platform for localization of the capture probe on the electrodes. In an embodiment, the electrodes can be a carbon-based electrode printed on a cellulosic substrate. In an exemplary embodiment, the cellulosic substrate can be paper. In an exemplary embodiment, the functionalization may be done with proteins selected from streptavidin and avidin. These protein compounds have extraordinarily high affinity for conjugating compounds like biotin and hence can provide a good localization of the capture probes on the electrodes.

In an embodiment, the sensor can further include a potentiostat arrangement that can include at least, one working electrode, one counter electrode and at least one reference electrode. The counter and reference electrodes can be coupled to the working electrodes for measurement of the electrical signal in form of dataset including measurement of voltage and current associated with the oxidation-reduction process. In an exemplary embodiment, the housing can be cuboid-shaped which enables space for the one or more components of the device.

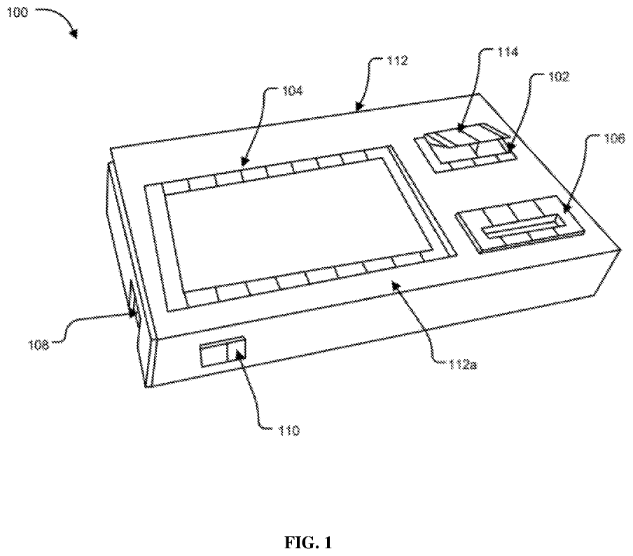

In an embodiment, the device can include a display screen to indicate the generated data. The display screen can be anyone selected from LCD screen or LED screen. The housing can further enclose a printed circuit board for various electrical connections between the one or more components. In an embodiment, the device can include a thermal printer for printing the generated data. FIG. 1 illustrates an exemplary biosensor device 100 in accordance with an embodiment of the present disclosure. The portable device 100 of FIG. 1 can include a housing 112 having an upper surface 112a. The housing 112 can be cuboid shaped as shown in FIG. 1. The device 100 can include a sensor cabinet 114 including a receptacle portion 102 to receive one or more sensors including working electrodes capable of oxidation-reduction process. The sensor can be configured to receive the biological sample and provide a surface for immobilization of a set of hybridization probes that form a three-dimensional complex upon independent hybridization with a target nucleic acid sequence of a gene corresponding to the microorganism in the biological sample to be identified, wherein the sensor upon being placed in the sensor cabinet 114 enables detection of the three-dimensional complex by measurement of an electrical signal due to the oxidation-reduction process at the working electrodes, wherein the electrical signal may be measurable by the device that may facilitate the identification of the microorganism in the biological sample in DNA-based detection of any or a combination of an infection and an antimicrobial resistance caused by the microorganism capture probe and a detector probe. The cabinet can enable to secure the sensor within the housing for detection purpose, wherein the sensor cabinet can also avoid spillage of any sample/reagents during experiment and enables to keep the surface steady for obtaining good precision. The sensor can include a potentiostat arrangement including at least one counter electrode and at least one reference electrode that are coupled to the working electrodes for measurement of the electrical signal in form of dataset including measurement of voltage and current associated with the oxidation-reduction process. The device 100 can include a LED display screen 104 to view the generated data and a thermal printer 106 to print the generated date to give immediate results.

Based on the requirements, the display screen can be used to display one or more aspects of the analysis i.e. for result description, for displaying the graph and excel data of species-specific infection and drug resistance as well as other details of the analysis. In one embodiment, multiple onboard LED may be present to enable the indication/display of results. The thermal printers 106 may enable to obtain quick printed form of data/results to the physician and patient, especially in remote rural areas wherein getting access to such facilities may be challenging and practically difficult. The data can also be transferred to a laptop or computer via a USB port 108. The device 100 can also have a power button 110 for charging/connecting to a power source. In an embodiment, the device 100 can also be powered with a rechargeable battery. In addition to the components shown, the device 100 can also have a control panel which can provide control to one or more menu buttons or switch on/off option.

The device can include a printed circuit board (PCB) wherein the PCB can be placed within the housing. The PCB can include LCD headers, potentiostat connections and for counter electrode (CE), reference electrode and working electrode respectively. In an exemplary embodiment, the PCB dimensions can be 100×60 mm whereas the complete device dimension can be 150×100×20 mm, although the embodiments of the present invention are not limited by the mentioned size/dimensions. The PCB can also include connection points for power connectivity and USB port respectively. The PCB can include mounting holes for mounting the PCB within housing of the device. Thus, the device is a complete and robust solution which can detect microbial infection and drug resistance and provide immediate result onscreen, which can also be physically available in terms of paper using thermal printer. Further, the biosensor DNA based technology can enable detection of infection and antibiotic resistance in less than 2 hours. The portable device can be used for infection drug resistance, which can be used to detect any infection i.e. hospital acquired infection, Viral infection, TB infection, sepsis and fungal infection and the like. Using specific capture and detector probe for wild strains, pathogenic strain, microbial infection in blood sample, urine sample and other biological fluid of body, it is possible to obtain fast, accurate and cost-effective detection of the microorganism. Using specific gene of accessory genome from genomic DNA or plasmid, antimicrobial resistance in blood and urine sample and other biological fluid of body can be known easily and effectively. Thus, the device is unique and a practically viable solution for detecting infection and antimicrobial resistance based on paper based microfabrication technology, which create can enable high sensitivity due to complex formed by double hybridization using specific probes.

In an embodiment, each of the first oligonucleotide and the second oligonucleotide can be single-stranded oligonucleotide complimentary to each strand of a pair of strands in a target nucleic acid sequence of a gene corresponding to the microorganism to be identified. Upon cell lysis of the microorganism, the target nucleic acid sequence may be accessed for independent hybridization with the detector probe and the capture probe. In one embodiment, upon cell lysis, the detector probe may be hybridized with one strand of the pair of strands of the target nucleic acid sequence to form a target nucleic acid-detector probe complex, followed by hybridization of the capture probe with another strand of the pair of strands of the target nucleic acid sequence to form a target nucleic acid-capture probe-detector probe complex, which may be a three-dimensional complex between the target nucleic acid, the capture probe and the detector probe.

In an embodiment, formation of three-dimensional complex can be detected by the one or more electrodes by the oxidation-reduction process that enables generation of an electrical signal for identifying the microorganism in the sample in DNA-based detection of the infection and antimicrobial resistance. In an embodiment, the detection of the formation of the three-dimensional complex can be done by using one or more reagents selected from any or a combination of anti-fluorescein monoclonal Fab fragment, horseradish peroxidase, a buffer, reagent for cell lysis and detection reagents. The reagent anti-fluorescein monoclonal Fab fragment may be used for detection of fluorescein-labelled compounds whereas reagent horseradish peroxidase is a metalloenzyme that can catalyse the oxidation of various organic substrates. This reagent may be added after formation of the three dimensional complex, wherein the anti-fluorescein monoclonal Fab fragment may interact with the fluorescein of the detector probe and enable detection of the three-dimensional complex. The reagent horseradish peroxidase may enable to promote the oxidation and reduction process to generate the electrical signal that may be measured using one or known techniques. Thus, these reagents may enable cell-lysis and/or measurement of one or more attributes related to qualitative and/or quantitative analysis of the three-dimensional complex for effective identification of the type of the microorganism and their quantitative analysis.

In certain embodiments the present disclosure provides a capture probe and a detector probe specific to a target nucleic acid sequence of a specific gene(s) for identifying a pathogenic bacteria type bacteria; a capture probe and a detector probe specific to virulent nucleic acid sequence of a specific gene, for identifying an antibiotic resistant bacterial strain, a multidrug resistant bacterial strain, an extensively drug-resistant strain; or pan drug-resistant strain, a capture probe and a detector probe specific to drug resistant nucleic acid of a specific gene was used. The set of capture probe and detector probe non-specific to any nucleic acid is used as a positive control (universal probe positive for gram negative and gram-positive bacteria)). Based on the type of oligonucleotide being used in the capture probe and the detector probe, the type of microorganism may be identified. The formation of the three-dimensional complex may be confirmed by detection using one or more methods that can enable measurement of electrical signal due to formation of the complex, wherein the three-dimensional complex formation may depend on the nature of the target nucleic acid sequence and thus provide a qualitative data on the type of microorganism being detected.

Based on the measurement, the current output can provide an estimate of hybridization between capture probe, target nucleic acid sequence and the detector probe, wherein the result can indicate number of bacterial cells in the sample that may be directly proportional to current output. In an embodiment, the current output may be measured by using voltammetry. In an exemplary embodiment, a potential of +2.5V may be applied for a time duration in the range of 30 seconds to 1.5 minutes with scan rate of 0.1 second to obtain the current output that may provide a quantitative idea regarding the number of bacterial cells in the sample.

In an embodiment, the detection of the sample for identifying the microorganism may be done in a time duration in the range of 1 min to 120 mins. In an exemplary embodiment, the detection may be done in a time period of less than 2 hours. In another exemplary embodiment, the sample may be detected for identification of the microorganism in the range of 30 seconds to 1.5 minutes. The sample may be a biological fluid selected from blood, urine and other biological fluids of a body. The device of the present disclosure may enable a detection limit in the range of 10¹ to 10¹⁰ CFU/ml in a sample such as blood and urine samples. The device may be very specific due to its gene specific probes hybridization to the target sequence.

In another aspect, the present disclosure provides a kit for identifying a strain of a gram negative bacterium in a sample for nucleic acid or nucleic acid based biosensor detection of an infection and antimicrobial resistance. The kit can include a cocktail mixture, a set of hybridization probes and one or more reagents, wherein the cocktail mixture may be capable of lysing the gram negative bacterium and releasing nucleic acid in a biological sample in a time period in the range of 20 to 40 minutes, wherein the cocktail mixture can include a combination of 1M sodium hydroxide solution, 10% Tween, 20 mM Tris (hydroxymethyl) aminomethane hydrochloride (Tris HCl), 1 mM EDTA, Lysozyme (10 mg/ml) in 10 mM tris HCL in a volume ratio of 1:1:1:0.5:0.25; wherein the one or more reagents selected from any or a combination of streptavidin, biotin, anti-fluorescein monoclonal Fab fragment, horseradish peroxidase, a buffer, reagents for cell lysis and detection reagents. The set of hybridization probes can include capture probes and detector probes complementary to and capable of hybridizing to a target nucleic acid sequences of a gene for identification of infection causing and drug resistant strain(s) of the microorganism selected from a wild type strain, an infection causing strain and an antibiotic resistant microorganisms.

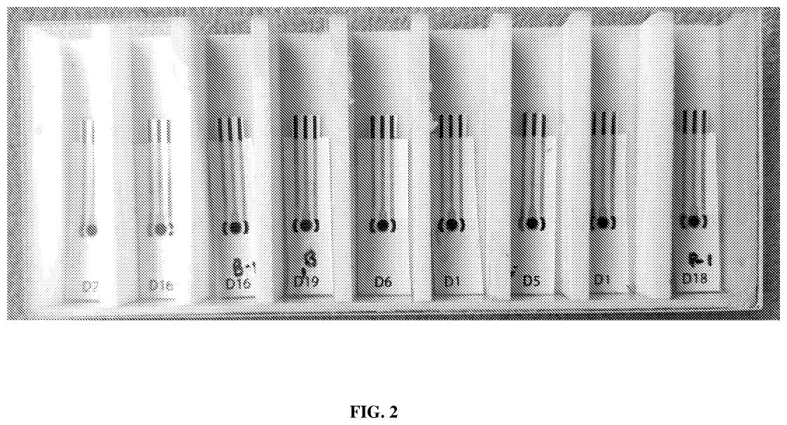

In an embodiment, the formation of the three-dimensional complex may be carried out inside an acrylamide cassette, wherein the sensor is placed inside the cassette to enable hybridization between the probes and the target nucleic acid sequence on the surface of the sensor. FIG. 2 illustrates an exemplary cassette, in accordance with an embodiment of the present disclosure. In an embodiment, the length of the cassette can be in the range of 15 cm to 20 cm and the breadth can be in the range of 5 cm to 10 cm. In an exemplary embodiment, the length and width of the cassette can be 18 cm and 7 cm, respectively. The bottom of cassette can be made up of opaque white colour acrylamide material and the cover of the cassette can be made up of transparent acrylamide material for better visibility and transparency. The cassette can have a panel having plurality of columns in each panel, wherein the space between two adjacent columns can be in the range of 0.1 cm to 0.6 cm, so that solution of each sensor will not spill out on each other. The length, width and depth of each column can be in the range of 1 cm to 5 cm. In an exemplary embodiment, the space between two adjacent column can be 0.5 cm, and the width, length and depth of each can be 1.5 cm, 5 cm and 1 cm respectively as shown in FIG. 2. is the cassette can have a good heat resistance, temperature tolerance >65° C. such that hybridization can take place in the cassette within an incubator. The screen-printed electrode can tend to be very thin and slippery on surface, and hence the surface can be made comparatively rough for ease of retrieval after the completion of experiment.

In an embodiment the hybridization between the fluorescein tagged single stranded oligonucleotide detector probe with the target nucleic acid sequence may be done at 65° C. in incubator for 10 minutes. The method in accordance with the present disclosure may be carried out by adopting cyclic voltammetry principle for measuring the reduction potential of a microbial species in the reaction solution. In an embodiment, the suitable equipment to measure the oxidation-reduction response may be portable potentiometer, which being, a low cost equipment can add to the cost-effectiveness of the testing.

In an embodiment the hybridization may be carried out by applying current to the electrodes. The potential applied can be of +2.5V for about 30 seconds to 1.5 minutes, preferably from about 1 minute to about 1.5 minutes with a scan rate of 0.1 second to about 0.5 seconds. The current output may impart threshold of hybridization between capture probe-target nucleic acid sequence-detector probe. The result may be generated in format of number of bacterial cells in the sample directly proportional to current output using voltammetry.

The localization of probes, and double hybridization between capture probe, target nucleic acid sequence and detector probe provide three-dimensional structure on screen printed electrode acting as a DNA biosensor, and thereby result in profound sensitivity in detecting infection causing and antibiotic resistant microorganisms and thereby enables detection of infection and antibiotic or multidrug resistance. The method in accordance with the present invention is capable of detecting infection causing and antibiotic resistant microorganisms within 1 minute to 120 minutes.

In an aspect, the present disclosure relates to a biosensor based system for identifying a strain of a gram negative bacterium in a sample for nucleic acid (or DNA) based detection of an infection and antimicrobial resistance. The system can be designed and customized for detecting a wide range of infection causing gram negative bacteria in a sample, wherein the system can be customized to act as a species specific probe or a universal probe. In an embodiment, the system can detect a microbial strain selected from wild type strain, a pathogenic strain, an antibiotic resistant bacterial strain, a multidrug resistant bacterial strain, a drug-resistant strain, and pan drug-resistant strain. The system of the present disclosure can be an effective alternative for most of the existing modalities in term of time, experimental-expenditure, human labor and cost for infection detection and antibiotic resistance to obtain the results in a qualitative and quantitative manner.

As per an embodiment of the present disclosure, the system can include a sensor including one or more carbon-based working electrodes capable of under going an oxidation-reduction process and a set of hybridization probes. The working electrodes may be screen printed on a paper and may be functionalized with at least one protein. The functionalized protein on the electrodes may provide a platform for localization of the capture probe on the electrodes. In an exemplary embodiment, the functionalization may be done with proteins selected from streptavidin and avidin. These protein compounds have extraordinarily high affinity for conjugating compounds like biotin (tagged to capture electrodes) and hence can provide a good localization of the capture probes on the electrodes.

The capture probe can include a first oligonucleotide that may be a single-stranded oligonucleotide. The first oligonucleotide may be tagged with biotin so that the capture probe can be localised on the working electrodes, wherein biotin is capable of conjugating with the protein on the electrodes. In an exemplary embodiment, biotin may be tagged at 5-′ or 3′-end of the first oligonucleotide. In an embodiment, the detector probe can include a second oligonucleotide that may be a single-stranded oligonucleotide tagged with fluorescein. In an exemplary embodiment, fluorescein that may be tagged at 5-′ or 3′-end of the second oligonucleotide.

In an embodiment, each of the first oligonucleotide and the second can be single-stranded oligonucleotide complimentary to each strand of a pair of strands in a target nucleic acid sequence of a gene corresponding to the strain of the gram negative bacterium to be identified. Upon lysis of the bacterium cell wall, the target nucleic acid sequence may be accessed for independent hybridization with the capture probe and the detector probe. In an embodiment, upon cell lysis, the detector probe may be hybridized with one strand of the pair of strands of the target nucleic acid sequence to form a target nucleic acid-detector probe complex, followed by hybridization of the capture probe with another strand of the pair of strands of the target nucleic acid sequence to form a target nucleic acid-capture probe-detector probe complex, which may be a three-dimensional complex between the target nucleic acid and the set of hybridization probes.

In an embodiment, formation of three-dimensional complex can be detected by the one or more electrodes by the oxidation-reduction process that enables generation of an electrical signal for identifying the strain of the gram negative bacterium in the sample in DNA-based detection of the infection and antimicrobial resistance. In an embodiment, the detection of the formation of the three-dimensional complex can be done by using one or more reagents selected from any or a combination of anti-fluorescein monoclonal Fab fragment, horseradish peroxidase, a buffer and detection reagents. The reagent anti-fluorescein monoclonal Fab fragment may be used for detection of fluorescein-labelled compounds whereas reagent horseradish per oxidise is a metalloenzyme that can catalyse the oxidation of various organic substrates. These reagents may be added after formation of the three-dimensional complex, wherein the anti-fluorescein monoclonal Fab fragment may interact with the fluorescein of the detector probe and enable detection of the three-dimensional complex. The reagent horseradish peroxidase may enable to promote the oxidation and reduction process to generate the electrical signal that may be measured using one or known techniques. Thus, these reagents may enable measurement of one or more attributes related to qualitative and/or quantitative analysis of the three-dimensional complex for effective identification of the type or the strain of the bacterium and their quantitative analysis.

In certain embodiments the present disclosure provides a capture probe and a detector probe specific to a target nucleic acid sequence of a specific gene(s) for identifying a specific bacteria; a capture probe and a detector probe specific to virulent nucleic acid sequence of a specific gene for identifying a pathogenic bacteria; a capture probe and a detector probe specific to a resistant nucleic acid sequence of DNA/Plasmid for identifying an antibiotic resistant bacterial strain, a multidrug resistant bacterial strain, an extensively drug-resistant strain; or pan drug-resistant strain. In an embodiment, the set of probes including capture probe and detector probe non-specific to any nucleic acid (positive for gram negative and gram-positive bacterial species) may be used as a positive control.

Based on the type of oligonucleotide being used in the capture probe and the detector probe, the type of bacterial strain may be identified. The formation of the three-dimensional complex may be confirmed by detection using one or more methods that can enable measurement of electrical signal due to formation of the complex, wherein the three-dimensional complex formation may depend on the nature of the target nucleic acid sequence and thus provide a qualitative data on the type of bacterium being detected. Based on the measurement, current output can provide an estimate of hybridization between capture probe, target nucleic acid sequence and the detector probe, wherein the result can indicate presence of the bacterium in a biological sample, wherein the number of bacterial cells in the sample may be directly proportional to current output. In an embodiment, the current output may be measured by using cyclic voltammetry. In an exemplary embodiment, a potential of +2.5V may be applied for a time duration in the range of 30 second to 1.5 minutes with scan rate of 0.1 second to obtain the current output that may provide a quantitative idea regarding the number of bacterial cells in the sample.

In an embodiment, the system of the present disclosure can be used for identifying antibiotic resistant strain selected from wild type strain, an antibiotic resistant bacterial strain, a multidrug-resistant (MDR), extensively drug-resistant (XDR), and pan drug-resistant (PDR) strains of the gram negative bacteria selected from E. coli, Klebsiella pneumoniae and Pseudomonas aeruginosa.

In an embodiment, the set of hybridization probes may be capable of individually hybridizing to any of the target nucleic acid sequence of the gene selected from CTX-M, KPC gene, and NDM-1 gene of E. coli, Klebsiella pneumoniae and Pseudomonas aeruginosa strains. Klebsiella pneumoniae is one of common nosocomial pathogens causing urinary tract infections, bacteria, and pneumonia in all over world. Carbapenems are a class of β-Lactam antibiotics with a broad spectrum of antibacterial activity. Misuse, overuse and abuse of the carbapenems can increase resistance in the K. pneumoniae. KPC—(Klebsiella pneumoniae carbapenemase-) producing K. pneumoniae strains are the most common carbapenamase-producing pathogens worldwide. The system of the present disclosure may also enable identification of the resistant strains to antibiotics selected from carbapenems and amino glycosidase and colistin in detection of any or a combination of multi-drug resistance, extensively drug-resistance and pan drug-resistance.

In another embodiment, the set of hybridization probes may be capable of individually hybridizing to any of the target nucleic acid sequence of the gene selected from CTX-M, KPC and NDM-1 gene, of E. coli, Klebsiella pneumoniae and Pseudomonas aeruginosa. The system of the present disclosure may also to identify the strain resistant to antibiotics selected from carbapenem, amino glycosidase and colistin in detection of any or a combination of multi-drug resistance, extensively drug-resistance and pan drug-resistance. Pseudomonas aeruginosa may be considered as one of the top five microorganisms causing severe infection, wherein its bacterial surface factors such as flagella, pili and lipopolysaccharide as well as active processes such as the secretion of toxins, biofilm formation, and quorum sensing are virulence determinants that impact the outcome of infections caused by the bacterial strain. In an embodiment, the system of the present disclosure can detect pathogenic strain using 16sRNA genes for pathogenic Pseudomonas aeruginosa identification, whereas detection of NDM gene may be done to confirm resistance to antibiotics including carbapenem, amino glycosidase and colistin that may suggest detection of an extensively drug-resistant Pseudomonas strain.

In an embodiment, the set of hybridization probes may be capable of individually hybridizing to 16S rRNA of Pseudomonas aeruginosa and the probes may be used as marker to identify the pathogenic and discriminating from the pathogenic and drug-resistant strains of Pseudomonas aeruginosa.

In an embodiment, the set of hybridization probes may be capable of individually hybridizing to any of the target nucleic acid sequence of the gene CTX-M, selected from extended spectrum beta-lactamase (ESBL) producing E. coli resistant to antibiotics selected from cephalosporins belonging to one to four classes, fluoroquinolones, and monobactam in detection of any or a combination of multi-drug resistance. In another embodiment, the set of hybridization probes may be capable of individually hybridizing to a target nucleic acid sequence of 16s RNA gene of E. coli and identifying the pathogenic E. coli strain to detect the infection. In another embodiment the present disclosure provides probes capable of individually hybridizing to a target nucleic acid sequence of blaCTXM-1 of E. coli and identifying the drug resistant strain, thereby detecting drug resistance.

In an embodiment, for pan drug resistance confirmation, three strains of E. coli, Klebsiella pneumoniae and Pseudomonas aeruginosa may be done that can show combination of CTX-M1, KPC and NDM-1, genes presence. Thus, a wide variety of MDR, XDR and PDR bacterial strains can be identified by the system of the present disclosure thus making the system versatile and effective as well as a fast detection technique than the conventional counter parts.

In an embodiment, the first oligonucleotide sequence (capture probe) and the second oligonucleotide sequence (detector probe) can be selected from SEQ ID No. 1 (CTGCGGGTAACGTCAATGAGCAAA), SEQ ID No. 2 (GGTATTAACTTTACTCCCTTCCTC), SEQ ID No. 3 (CTATTACTAC AGGTGAAGGT GGAAT), SEQ ID No. 4 (TCACGAATGA CAAAACACTT TATGA), SEQ ID No. 5 (CCATGAAGTC GGAATCGCTA GTAA), SEQ ID No. 6 (TAGATCAGAA TGCTACGGTG AATACG), SEQ ID No. 7 (CTGATACTGA CACTGAGGTG CGAAA), SEQ ID No. 8 (CGTGGGGAGC AAACAGGATT AGATA), SEQ ID No. 9 (GGCATTGATT AACACAGCAG ATAA), SEQ ID No. 10 (CAAATACTTT ATCGTGCTGA TGAGC), SEQ ID No. 11 (GTTTAATGTT GGAGGCTAAG TGATA), SEQ ID No. 12 (ACAGTAAGGA CGCATACAAT AATAAG), SEQ ID No. 13 (ATGTCACTGA ATACTCGTCC TAGAA), and SEQ ID No. 14 (CGTTAGATTG GCTTACACCA TTAGA).

In an embodiment, the first oligonucleotide sequence (capture probe) can be selected from SEQ ID No. 1, SEQ ID No. 3, SEQ ID No. 5, SEQ ID No. 7, SEQ ID No. 9 and SEQ ID No. 11.

In an embodiment, the second oligonucleotide sequence is selected from SEQ ID No. 2, SEQ ID No. 4. SEQ ID No. 6, SEQ ID No. 8 SEQ ID No. 10 SEQ ID No. 12 and SEQ ID No. 14.

In an exemplary embodiment, for detection of E. coli, the second oligonucleotide sequence (capture probe) can have a SEQ ID NO. 1, and the detector probe can have SEQ ID NO. 2. In another exemplary embodiment, for detection of rfb-gene for E-coli, the second oligonucleotide sequence (capture probe) can have a SEQ ID NO. 3, and the detector probe can have SEQ ID NO. 4. In another exemplary embodiment, for detection of Klebsiella pneumoniae, the second oligonucleotide sequence (capture probe) can have a SEQ ID NO. 5, and the detector probe can have SEQ ID NO. 6. In another exemplary embodiment, for detection of Pseudomonas aeruginosa, the second oligonucleotide sequence (capture probe) can have a SEQ ID NO. 7, and the detector probe can have SEQ ID NO. 8. In another exemplary embodiment, for detection of KPC-2 gene for antibiotic resistance (Klebsiella Pneumoniae) detection sensor, the second oligonucleotide sequence (capture probe) can have a SEQ ID NO. 11, and the detector probe can have SEQ ID NO. 12. In another exemplary embodiment, for detection of NDM-1 gene for antibiotic resistance (Pseudomonas aeruginosa), the second oligonucleotide sequence (capture probe) can have a SEQ ID NO. 13, and the detector probe can have SEQ ID NO. 14.

In an embodiment, the system can be used for specific detection of a strain of a gram negative bacteria. In another embodiment, the system can be used to probe more than one strain of bacterium thereby enabling to be used as a universal probe.

In certain embodiments the present disclosure provides a capture probe and a detector probe specific to a target nucleic acid sequence of a specific gene(s) for identifying antibiotic resistance selected from wild strain, pathogenic strain and an extended spectrum beta-lactamase (ESBL) producing strains of E. coli in patients with acute leukaemia or other such diseases.

In an embodiment, the set of hybridization probes may be capable of individually hybridizing to 16S rRNA of pathogenic E. coli strain MTCC-4296, wherein the probes may be used to identify a UTI infection and discriminate from the pathogenic and drug-resistant E. coli strains.

In an embodiment, the set of hybridization probes may be capable of individually hybridizing to a target nucleic acid sequence of rfb-E gene of E. coli O157:H7 strain and identifying the pathogenic E. coli strain (Enterohemorrhagic E. coli-EHEC) to detect haemorrhagic infection.

In an embodiment, the set of hybridization probes may be capable of individually hybridizing to any of the target nucleic acid sequence of the gene selected from CTX-M1, in extended spectrum beta-lactamase (ESBL) producing E. coli resistant to antibiotics selected from cephalosporins belonging to one to four classes, fluoroquinolones, and monobactam in detection of multi-drug resistance.

In an embodiment, the detection of the sample for identifying the strain of E. coli may be done in a time duration in the range of 1 min to 120 mins. In an exemplary embodiment, the detection may be done in a time period of less than 2 hours. In another exemplary embodiment, the sample may be detected for identification of the strain of E. coli in the range of 1 minutes to 5 minutes. The sample may be a biological fluid selected from blood, urine and other biological fluids of a body. The system of the present disclosure may enable a detection limit in the range of 10 to 10¹⁰ CFU/ml in a sample such as blood and urine samples. The system may be very specific due to its gene specific probes hybridization to the target sequence.

The detecting system in accordance with the present disclosure is novel, rapid, economical, sensitive and specific to target nucleic acid of a specific gene, robust, portable and capable of being used as a point of care device. The detecting system in accordance with the present disclosure renders it useful specifically in oncologist clinics to detect infection and antibiotic resistance status. The detecting system in accordance with the present disclosure can enable a physician to identify the E. coli infection and antibiotic resistance in a given sample in acute leukaemia (AL) patients during induction therapy, which will help in stratifying the AL patients according to their disease severity to tolerate the treatment. Thus, the detecting system in accordance with the present disclosure can help physician to and arrive at the treatment regimen by selecting suitable antibiotics according to the site and species-specific infection. The detecting system in accordance with the present disclosure can be useful in detecting infection in patients with different malignancies, which are predominant in bacterial infection and likewise in patients with others diseases as well. The detecting system in accordance with the present disclosure can be used as a monitor tool of infection in patients suffering from acute leukaemia, other cancers and diseases.

In another aspect, the present disclosure provides a method for preparation of the electrodes and probes for identification of infection-causing bacterial strain in a given sample. The method can include obtaining electrodes by micro fabrication technology by printing carbon-based electrodes on paper, wherein the electrodes may be capable of undergoing and measuring oxidation-reduction; providing a set/panel of a single-stranded oligonucleotide capture probe, and a single-stranded oligonucleotide detector probe, each probe bearing oligonucleotides that can be complimentary to and capable of hybridizing to target nucleic acid sequence of a specific gene(s) of one or more of a wild type, infection causing and antibiotic resistant bacterial cells; functionalizing printed carbon-based electrodes with streptavidin; localizing a single-stranded oligonucleotide capture probe tagged with biotin on the DNA biosensor; lysing the bacterial cells in the given sample and releasing the target nucleic acid sequence; allowing the first hybridization to take place between a fluorescein tagged single stranded oligonucleotide detector probe capable of hybridizing with the target nucleic acid sequence at a specific temperature to form a detector probe-target nucleic acid complex; allowing the second hybridization to take place between the detector probe-target nucleic acid complex with the capture probe localized on the screen printed electrode-target nucleic acid sequence forming a three dimensional structure of the target nucleic acid sequence between the capture probe and the detector probe towards the sensor; adding anti-fluorescein monoclonal Fab fragment on the resultant three dimension structure of the capture probe-target nucleic acid sequence-detector probe; adding horseradish peroxidase; and measuring the oxidation-reduction response at the sensor electrodes on a suitable test equipment.

In an embodiment the hybridization between the fluorescein tagged single stranded oligonucleotide detector probe with the target nucleic acid sequence may be done at 65° C. in an incubator for 10 minutes. The method in accordance with the present disclosure may be carried out by adopting cyclic voltammetry principle for measuring the reduction potential of a microbial species in the reaction solution. In an embodiment, the suitable equipment to measure the electrical signal/the oxidation-reduction response may be portable potentiostat/potentiometer, which being, a low-cost equipment can add to the cost-effectiveness of the testing.

In an embodiment the hybridization may be carried out by applying current to the electrodes. The current applied can be of +2.5V for about 30 seconds to 1.5 minutes, preferably from about ≤1 minute with a scan rate of 0.1 second to about 0.5 seconds. The current output may impart threshold of hybridization between capture probe-target nucleic acid sequence-detector probe. In an exemplary embodiment, the result may be generated in format of number of bacterial cells in the sample directly proportional to current output using suitable test equipment.

The localization of probes, and double hybridization between capture probe, target nucleic acid sequence and detector probe provide three-dimensional structure on screen printed electrode acting as a nucleic acid based or DNA biosensor, and thereby result in profound sensitivity in detecting infection causing and antibiotic resistant bacterium and thereby enables detection of infection and antibiotic or multidrug resistance. The method in accordance with the present invention is capable of detecting infection causing and antibiotic resistant bacteria within 1 minute to 120 minutes.

The device, system, cassette and kit in accordance with the present disclosure is capable of detecting microbial cells from 10¹ to 10¹⁰ CFU/ml from a given sample, the sample preferably being blood and urine samples or any other biological body fluid. The device and kit of the present disclosure may be capable of providing results in terms of number of bacterial cells causing infection and antibiotic resistant in the sample, are accordingly more sensitive up-to >98% as compared to the conventional culture tools up-to 26% and method used to detect infection. Further, due to ability to identify specific bacterial cells due to specificity to target nucleic acid, the device, kit and the method of the present disclosure are contemplated to be superior to the existing PCR and sequencing tools and techniques. The device, and kit provided in accordance with the present disclosure can be used by a physician to prescribe specific antibiotic course to patients who are suffering from infection based on the output received. The device and kit of the present disclosure can also be used by a physician to identify infection in each and every disease where it can be used as monitoring tool to treat infection, thus providing faster and cost-effective analysis that can overcome the disadvantages of the conventional systems.

### EXAMPLES

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Example-1—Biosensor Device (Experimental Details of Sample Preparation and Measurement)

We developed 6 step quick technology based biosensor to detect infection and antimicrobial resistance using paper-based portable device to detect infection and antibiotic resistance in form of reduction current during oxidation reduction reaction it will complete within 2 hour and the steps are as follows.

Experiment was done inside the cassette, as shown in FIG. 2. First of all, screen-printed electrodes (SPE) was placed inside the acrylamide cassette and then the following steps were followed:
Step-1: functionalization of screen-printed electrodes (SPE) or sensors was done with streptavidin
Step-2: Immobilization of capture probe was done on the SPE
Step-3: A biological sample containing human urine (or blood sample) was taken from an acute leukaemia patient. The urine sample was used in undiluted form whereas a blood sample was diluted with water in 1:1 ratio before usage. Initially, the sample was centrifuged at 14000 rpm for 15 minutes at room temperature to obtain a sedimented portion/pellet of DNA/plasmid DNA which was used for further analysis whereas the supernatant was discarded. Further, a 350 μL cocktail mixture was prepared for adding to the pellet obtained from the urine sample for carrying out cell lysis of the gram negative bacterium to be detected. The cocktail mixture included 1 molar sodium hydroxide solution, 10% Tween+20 mM Tris (hydroxymethyl) aminomethane hydrochloride (Tris HCl), 1 mM EDTA, Lysozyme (10 mg/ml) in 10 mM tris HCL, wherein the volume ratio of all the five ingredients was 1:1:1:0.5:0.25. Upon adding the cocktail mixture to the pellet obtained by the centrifugation of the urine sample, the urine sample released target nucleic acid sequence after cell lysis. The resultant mixture was centrifuged at 14000 rpm for 15 minutes and the resultant supernatant solution was taken. And the first hybridization was allowed to take place between released nucleic acid (supernatant solution) and detector probe at 65° C. in incubator for 10 minutes.
Step-4: Second hybridization between capture probe, DNA/Plasmid DNA and detector probe was done inside a cool 4° C. box takes place at 65° C. in incubator for 15 minutes to form a three dimensional complex.
Step-5: Addition of anti-fluorescein monoclonal Fab fragment was done
Step-6: The sensor (with the 3-D complex) was taken out from cassette and placed into the sensor cabinet present within the housing (cuboid box) of the device (FIG. 1), where electrical connection for electrodes (Working, reference and counter) was already available. This was followed by addition of detection reagents on the sensor and reading of the sample on test instrument/device.

Using specific capture and detector probe for wild strains, pathogenic strain, microbial infection and antimicrobial resistance was reported in blood and urine sample and other biological fluid of body.

The sensitivity of the sensor was the result of location of probes hybridization. The current out-put gave an indication of the threshold of hybridization between capture probe-target sequence—and detector probe. The result was obtained in format of no. of bacterial cells in blood/urine directly proportional to current output. Potential of ±2.5 was applied for 30 see to 1.5 minutes with scan rate of 0.1 second. Detection limit of our sensor was in the range from 10¹ to 10¹⁰ CFU/ml from blood and urine samples. This paper-based sensor was very specific due to its gene specific probes hybridization to the target sequence.

The sensor device was used to detect a biological sample such as urine (undiluted form) or blood (dilution with water in 1:1 ratio). Using the present device, better sensitivity in data was obtained. As showed in table-1, existing test methods have limitations, i.e. optical density (OD) measurement can predict growth only in a range up to 10¹ to 10³ and hence for further downline experiments, samples need to be diluted for culture purpose. While culture can be sensitive method, but still if when we observe it closely, culture results can be read only between 10⁴ to 10⁷ CFU (colony forming unit)′ and hence it is difficult to measure beyond CFU value of 10⁷ and moreover it take almost 2-3 days' time, to finish the whole experiment using conventional methods. So overall sensitivity of existing gold standard method culture was found to be 24-26% whereas the present disclosures>98% sensitive. The device of the present disclosure can give results up to 10¹-10 CFU within 2 hours so it quite sensitive and fast in compare to existing modalities. Moreover, the device does not require high manpower or skill-set for usage, you can directly take blood or urine, process it on our sensor, using our technology, you will get gene specific bacterial infection and antibiotic resistance results in 90 minutes.

TABLE 1
Comparison of conventional techniques with the present device
Compare table of Culture and sensor technology

| Dilution | CFU | OD | current |
|---|---|---|---|
| 10-1 | Not countable (NC >3000) | 0.196 | −32.04 |
| 10-2 | NC | 0.016 | −25.61 |
| 10-3 | NC | Negative | −24.312 |
| 10-4 | NC | Negative | −23.0598 |
| 10-5 | 312 | Negative | −19.3524 |
| 10-6 | 80 | Negative | −15.8873 |
| 10-7 | 10 | Negative | −11.624 |
| 10-8 | Invisible | Negative | −9.75945 |
| 10-9 | Invisible | Negative | −8.99178 |
| Blank | Invisible | Negative | −0.002 |

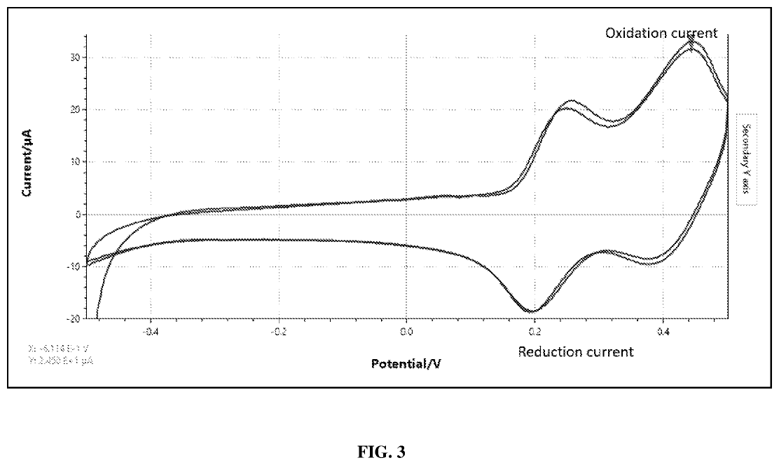

Using the device, detection result was obtained in format of no. of bacterial cells in blood/urine that were directly proportional to current output using portable tailored potentiostat for POC device for sensor technology. Potential of +2.5V was applied for 30 seconds to 1.5 minutes with scan rate of 0.1 second to obtain the voltammetry data as shown FIG. 3, wherein the presence of reduction current indicated detection/presence of microorganism whereas the type of probes for which the results tested positive gave confirmation on the type of microorganism thereby enabling identification of the microorganism, which in case of FIG. 3 related experiment was E. Coli. The detection limit of the sensor in the device was found to be 10¹ to 10¹⁰ CFU/ml from blood and urine samples. The paper based sensor was very specific due to its gene specific probes hybridization to the target sequence.

The workable range of the present device is as illustrated in Table 2:

TABLE 2
Workable range of the present device

| Output | Range |
|---|---|
| Applied DC potential range | ±2.5 V |
| Current range | 1-700 μA |
| DC Potential resolution | 0.8 mV |
| DC Potential accuracy | 0.08% of the scale |
| Result analysis time | 30 sec-90 sec |

### General Example-2 Formation and Detection of Complex Between Target Nucleic Acid Sequence and the Set of Hybridization Probes

### Step 1: Immobilization of Capture Probe

An electrode for biosensor-based detection system was prepared for detecting pathogens (E. coli, Klebsiella pneumonia and Pseudomonasaeruginosa) by microfabrication technology by printing carbon-based electrodes on paper. The printed carbon-based electrodes were functionalized with 10 μL of 0.5 mg/ml streptavidin at room temperature for 10 minutes inside a biosensor cassette. 10 μL of species specific targeted capture probe (E. coli, Klebsiella pneumonia and Pseudomonas aeruginosa) was immobilized on the screen printed electrode at room temperature for 30 minutes, after which the probe was then washed with phosphate buffer saline (having 19 ml of 1M Na₂HPO₄ and 81 ml of 1M K₂HPO₄) having pH of 7.4.

### Step 2: Nucleic Acid Release and Formation of a First Complex Between Target Nucleic Acid Sequence of Biological Sample and Detector Probe (First Hybridization)

A biological sample containing human urine was taken. 1 ml urine sample was centrifuged at 14000 rpm for 15 minutes at room temperature to obtain a sedimented portion/pellet which was used for further analysis whereas the supernatant was discarded. Further a 350 μL cocktail mixture was prepared for adding to the pellet obtained from the urine sample for carrying out cell lysis of the gram negative bacterium to be detected. The cocktail mixture included 1 molar sodium hydroxide solution. 10% Tween+20 mM Tris (hydroxymethyl) aminomethane hydrochloride (Tris HCl). 1 mM EDTA. Lysozyme (10 mg/ml) in 10 mM tris HCL, wherein the volume ratio of all the four ingredients was 1:1:1:0.5:0.25. Upon adding the cocktail mixture to the pellet obtained by the centrifugation of the urine sample, the urine sample released target nucleic acid sequence after cell lysis. The resultant mixture was centrifuged at 14000 rpm for 15 minutes and the resultant supernatant solution was taken. A complex based hybridization between nucleic acid sequence and detector probe was obtained by adding a species specific detector probe to the supernatant solution inside a 65° C. incubator for 10 minutes. The detector probe was designed and prepared by tagging a single-stranded oligonucleotide with fluorescein. A first hybridization was allowed between a fluorescein tagged single stranded oligonucleotide detector probe and the target nucleic acid sequence at 65° C. for 10 minutes to form a detector probe-target nucleic acid complex.

### Step 3: Formation and Detection of a Second Complex Formed Between the First Complex Obtained in Step-2 and the Capture Probe on the Working Electrode (Second Hybridization)

After first hybridization between released nucleic acid and species specific detector probe, the complex (detector probe-target nucleic acid complex) was allowed to undergo second hybridization with the capture probe localized on the screen printed electrode as prepared in the step-1, that lead to formation of a three dimensional structure of the target nucleic acid sequence with the capture probe and the detector probe in 4° C. box at inside a 65° C. incubator for 15 minutes. To this 3-dimensional complex on the electrode, reagents such as 10 μL anti-fluorescein monoclonal Fab fragments in 1 ml 0.5% casein was added for 10 minutes and further washed with 1 ml PBS to remove unwanted binding. Further 50 μL horseradish peroxidase—TMB substrate was added to the electrode with the 3-dimensional complex to measure the oxidation-reduction response at the electrodes in term of current at constant voltage between (±2.5V).

In summary, a whole panel of key infection and drug resistant bacteria detection sensor was developed, that are major cause of most of the infection and drug resistance of several disease, specially in AL, immune compromised patients and the like.

### Example 3: Detection of Infection Causing Bacterium

a) Detection of Infection Caused by E. coli Strain Infection Sensor:

For purpose of detection of infection caused by E-coli, a specific strain of E-coli i.e. MTCC 4296 was used that was purchased from Microbial Type Culture Collection and Gene Bank. India. The oligonucleotide sequence corresponding to E. coli infection sensor used was as follows: Capture probe SEQ ID NO. 1 and Detector probe SEQ ID NO. 2.

This bacterial culture was grown in Luria broth over night at 37° C. at 180 rpm in an incubator cum shaker. The cells were isolated in log phase where OD between (0.5-1) at 600 nm was used, and further spiked individually with blood and urine sample wherein the urine sample was used in undiluted form whereas blood sample was diluted with water in 1:1. The sensor was standardized for E. coli in culture using standard E. coli bacterial isolates and later implemented in experiment for detection in the blood and urine spiked with E. coli strain up to 10¹ to 10¹⁰ CFU. Same protocol was followed for rest of sensor development.

### b) Detection of Infection Caused by Klebsiella Pneumonia Infection Sensor:

Same protocol was followed as in experiment (a) except that Klebsiella Pneumonia strain was used from a repository at Indian Institute of Technology using specific probes. The oligonucleotide sequence corresponding to Klebsiella pneumoniae sensor used was as follows: Capture probe SEQ ID NO. 5 and Detector probe SEQ ID NO. 6.

c) Detection of Infection Caused by Pseudomonas aeruginosa Infection Sensor:

Same protocol was followed as in experiment (a) except that Pseudomonas aeruginosa strain (NCIMB-8626) was used that was from purchased from NuLife Consultants & Distributors (Pvt.) Ltd., New Delhi India. The oligonucleotide sequence corresponding to Pseudomonas aeruginosa used was as follows: Capture probe SEQ ID NO. 7 and Detector probe SEQ ID NO. 8.

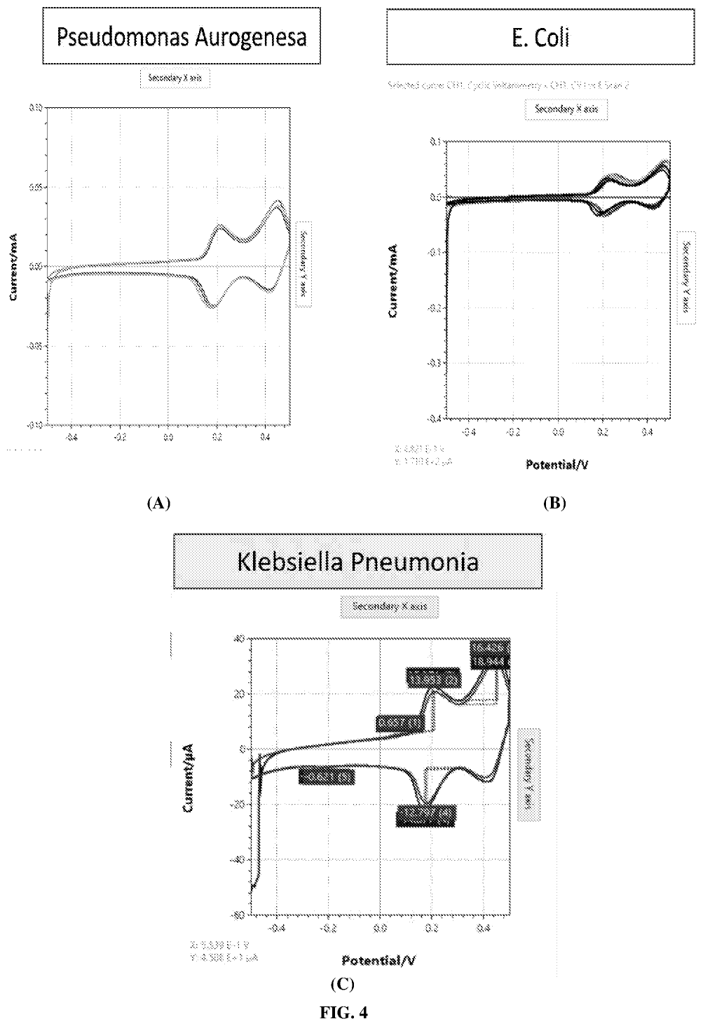

The sensing results for all the 3 strains (Pseudomonas aeruginosa, E. coli and Klebsiella pneumonia) were obtained by cyclic voltammetry as provided in FIGS. 4A-4C as well as in Table 3 below

TABLE 3
Cyclic voltammetry results in detection of infection caused
by gram negative bacterium
Pathogenic strains of Pseudomonas aeruginosa, E. coli and
Klebsiella pneumonia

| Pseudomonas aeruginosa | | E. coli | | Klebsiella Pneumonia | |
|---|---|---|---|---|---|
| Voltage | Current-μA | Voltage | Current-μA | Voltage | Current-μA |
| 0.15 | −25.2 | 0.17 | −24 | 0.22 | −22 |
| 0.16 | −22.7 | 0.19 | −21 | 0.21 | −25 |

### Example 4: Detection of Antibiotic Resistance

The antibiotic sensitivity of gram negative bacteria was tested by employing the Kirby-Bauer's technique as suggested by the Clinical & Laboratory Standards Institute (CLSI) guidelines.

i. Pseudomonas Drug Resistance Sensor

For the Pseudomonas aeruginosa drug resistance (NDM) sensor, clinical sample was grown and validated in our lab using culture for MBL (Metallo beta lactamase production), which was confirmed by disc diffusion and estrip (Estrip is E-test (imipenem 0.002-32 μg/ml and colistin 0.064-1024 μg/ml) was conducted based on the guidelines of the manufacturer). The tests were considered positive for imipenem and colistin when the ratio was ≥8 μg/ml. The E-test method was used to specify the minimum inhibitory concentrations (MICs) of imipenem and colistin (for colistin, MIC was detected only in resistant isolates by using Kirby-Bauer's technique). Further MBL (Metallo beta lactamase) producing Pseudomonas aeruginosa antibiotic Resistant clinical strain (NDM gene resistant) was subjected to antibiotic susceptibility tests that were performed on Mueller Hinton agar plates using standard method. The antibiotics used were Ceftazidime 30 μg/ml, Ciprofloxacin 5 μg/ml, Cefepime 30 μg/ml, Meropenem 10 μg/ml, Imipenem 10 μg/ml, colistin (10 μg), aztreonam (30 μg), levofloxacin (5 μg). The oligonucleotide sequence corresponding to Pseudomonas aeruginosa used was as follows: Capture probe SEQ ID NO. 13 and Detector probe SEQ ID NO. 14.

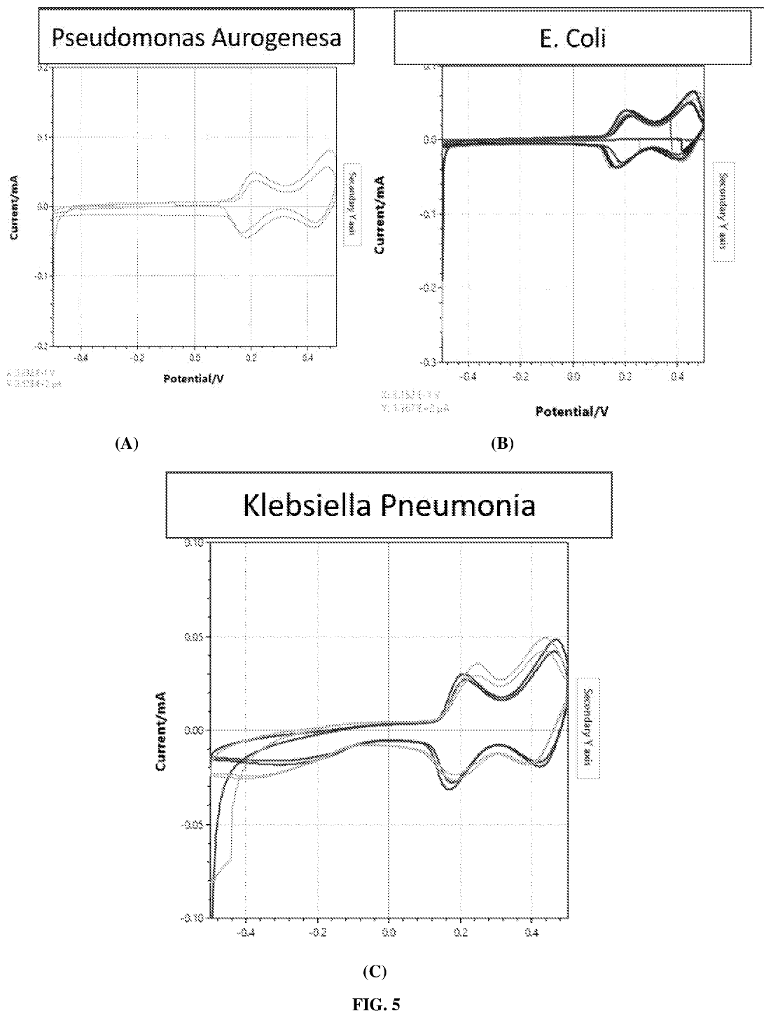

The detection results as measured by testing instrument (potentiostat arrangement of electrodes), wherein the results are as shown in FIG. 5A and Table 4.

ii. E. coli Drug Resistance Sensor:

E. coli drug resistance (CTX-M1) sensor was developed. ESBL producing E. coli drug resistant NCIM-2571 strain was purchased from NCL (National chemical Laboratory Pune, India). In the present experiment, the antibiotic sensitivity was tested for several class of antibiotics i.e. Cefotaxime 10 μg/ml, Rifampicin 50 μg/ml, Ciprofloxacin 1 μg/ml, Trimethoprim 0.5 μg/ml. The NCIM-2571 was used as marker for CTX-M resistance in ESBL producing E. coli during the sample analysis of biological sample. The results of the detection as derived by testing instrument (potentiostat arrangement of electrodes) is depicted in FIG. 5B. and Table 2.

iii. Klebsiella Drug Resistance Sensor:

To develop the Klebsiella drug resistance (KPC) sensor, carbepenamase resistant (KPC gene resistant) Klebsiella pneumonia MDR/XDR strain ATCC (BAA-1705) was purchased from NuLife Consultants & Distributors (Pvt.) Ltd., New Delhi India. The antibiotic sensitivity was tested using standard method for Klebsiella pneumonia MDR/XDR strain ATCC (BAA-1705) in incubator overnight at 37° C. in presence of antibiotics (Meropenem 10 μg/ml, Imipenem 10 μg/ml, Ceftazidime 30 μg/ml, Levofloxacin 5 μg/ml, Piperacillin-Tazobactam 100:10 μg/ml, Ciprofloxacin 5 μg/ml, Cefepime 30 μg/ml) with standard guideline. MDR/XDR strain ATCC (BAA-1705) was used as marker for KPC resistance in drug resistant Klebsiella pneumonia. The oligonucleotide sequence for detection KPC-2 gene for antibiotic resistance (Klebsiella Pneumoniae) used was as follows: Capture probe SEQ ID NO. 1 (GTTTAATGTT GGAGGCTAAG TGATA) and Detector probe SEQ ID NO. 1 (ACAGTAAGGA CGCATACAAT AATAAG). The detection results as measured by testing instrument (potentiostat arrangement of electrodes) are as shown in FIG. 5C and Table 4. The oligonucleotide sequence for detection of KPC-2 gene of Klebsiella Pneumoniae used was as follows: Capture probe SEQ ID NO. 11 and Detector probe SEQ ID NO. 12.

TABLE 4
Cyclic voltammetry results in detection of antibiotic resistance
caused by gram negative bacterium
Antibiotic resistant strains of Pseudomonas aeruginosa, E. coli and
Klebsiella pneumonia

| Pseudomonas aeruginosa | | E. coli | | Klebsiella Pneumonia | |
|---|---|---|---|---|---|
| Voltage | Current-μA | Voltage | Current-μA | Voltage | Current-μA |
| 0.16 | −34 | 0.16 | −23 | 0.16 | −23 |
| 0.18 | −29 | 0.17 | −26 | 0.17 | −20 |

### Detection of Gram Negative Bacteria Panel-Universal Probe (Panel of Hybridization Probes):

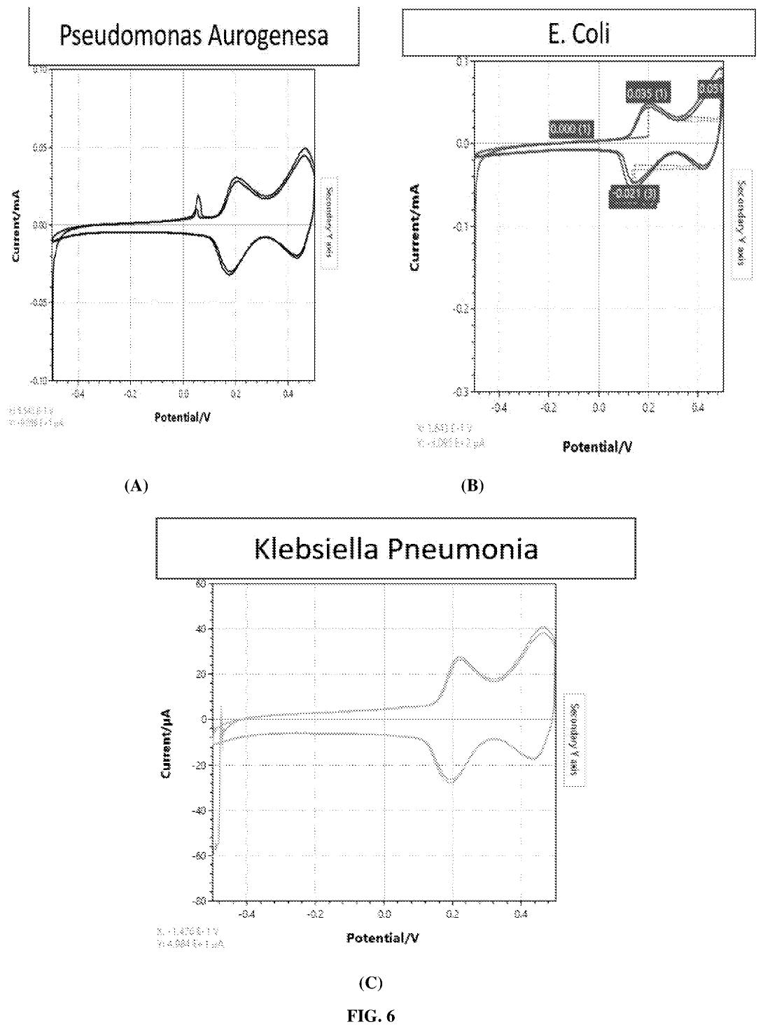

A panel of key dominating gram negative bacteria panel (E. coli, Klebsiella pneumonia, and Pseudomonas aeruginosa) was developed. For purpose of detection of either infection or antibiotic resistance, a specific strain of bacteria was used as positive control. A universal probe was developed usable as a positive control for all bacteria i.e. gram positive and gram negative bacteria and PBS was used as negative control for all the experiments as shown in FIGS. 6A-6C. The bacterial cultures were grown in Luria broth over night at 37° C. at 180 rpm in an incubator cum shaker as per standard guidelines. Cells were isolated in log phase where OD between 0.5-1 was used and further spiked with blood and urine sample, wherein the urine sample was used in undiluted form whereas blood sample was diluted with water in 1:1. After standardization of all sensors (Pseudomonas aeruginosa, E. coli, Klebsiella pneumonia) both for infection and antibiotic resistance in culture using standard bacterial isolates, experiment was repeated in blood and urine with spiking of bacterial strain up-to 10¹-10¹⁰ CFU. The results are summarized in FIGS. 6A-6C and in Table 5.

TABLE 5
Results for universal probedetection in different species

| Pseudomonas aeruginosa | | | | E. coli | | | | Klebsiella Pneumonia | | | |
|---|---|---|---|---|---|---|---|---|---|---|---|
| Pathogenic | | Resistant | | Pathogenic | | resistant | | Pathogenic | | resistant | |
| Voltage | | Voltage | | Voltage | | Voltage | | Voltage | | Voltage | |
| (V) | Current-μA | (V) | Current-μA | (V) | Current-μA | (V) | Current-μA | (V) | Current-μA | (V) | Current-μA |
| 0.17 | −28 | 0.17 | −22 | 0.21 | −23 | 0.14 | −28 | 0.13 | −28 | 0.19 | −19 |
| 0.17 | −25 | 0.16 | −21 | 0.20 | −22 | 0.13 | −26 | 0.17 | −25 | 0.19 | −17 |

The specificity of various probes/sensors (as described above) was also evaluated as provided in Tables 6 and 7 below. As observed in Table 5, the E-coli probe displays no prominent peaks at the specific voltages, as otherwise obtained using universal probe and K. Pneumonia probe. As observed in Table 7, the E-coli probe displays no prominent peaks at the specific voltages, as otherwise obtained using universal probe and Pseudomonas Aeruginosa probe. This indicates the precision and selectively/specificity of the K. Pneumonia probe as well as the workability/flexibility of the universal probe.

TABLE 6
Cyclic voltammetry results in evaluation of specificity
of Klebsiella probe in K. Pneumonia Strain

| E. coli probe in | | Universal probe | | K. Pneumonia probe | | K. Pneumonia probe in | |
|---|---|---|---|---|---|---|---|
| K. Pneumonia | | in K. Pneumonia | | in K. Pneumonia | | K. Pneumonia Strain | |
| Strain | | Strain | | Strain culture | | spiked urine | |
| Voltage | Current | Voltage | Current | Voltage | Current | Voltage | Current |
| (V) | (μA) | (V) | (μA) | (V) | (μA) | (V) | (μA) |
| 0.13 | −3 | 0.13 | −28 | 0.13 | −33 | 0.17 | −14 |
| 0.13 | −7 | 0.17 | −25 | 0.13 | −30 | 0.17 | −12 |
| | | 0.17 | −21 | 0.17 | −14 | 0.18 | −17.6 |

TABLE 7
Cyclic voltammetry results in evaluation of specificity of Pseudomonas Aeruginosa
probe in Pseudomonas Aeruginosa Strain

| | | Universal probe | | Universal probe | | Pseudomonas | | Pseudomonas | |
|---|---|---|---|---|---|---|---|---|---|
| E. coli probe in | | in Pseudomonas | | in Pseudomonas | | Aeruginosa probe | | Aeruginosa probe | |
| Pseudomonas | | Aeruginosa | | Aeruginosa | | in Pseudomonas | | in Pseudomonas | |
| Aeruginosa | | Strain | | Strain | | Aeruginosa Strain | | Aeruginosa Strain | |
| Strain | | (Pathogenic) | | (Resistant) | | (Pathogenic) | | (Resistant) | |
| Voltage | Current | Voltage | Current | Voltage | Current | Voltage | Current | Voltage | Current |
| (V) | (μA) | (V) | (μA) | (V) | (μA) | (V) | (μA) | (V) | (μA) |
| −0.21 | −0.0032 | 0.17 | −28 | 0.17 | −22 | 0.19 | −28 | 0.16 | −34 |
| −0.22 | −0.0042 | 0.17 | −25 | 0.16 | −21 | 0.20 | −30 | 0.18 | −29 |

In accordance with the experiments as elaborated herein above, it is clear that developing drug resistant sensor can prove to be a helpful tool for any physician. In particular. if a physician utilizes the system/sensor of the present invention, he/she may be able to understand/conclude about an infection or anti-biotic resistance as the presence of such an infection/resistance may be evident from a positive result (in terms of a signal as detected by cyclic voltammogram), for example, if the detection indicates NDM gene for Pseudomonas Aeruginosa in Blood or urine sample of patients, it may be useful to suggest based on the drug resistance of a patient for several class of antibiotics like Ceftazidime, Ciprofloxacin, Cefepime, Meropenem, Imipenem, colistin, aztreonam, levofloxacin. Further, drug resistant strains such as MDR/XDR/PDR bacteria can be easily detected using the present system/sensor, that allow immediate antibiotic treatment with resistant gene specific sensor based test. In addition, utilization of the present system/sensor also enables a physician to immediately initiate treatment (such as within 1-2 hours) after receiving the sample without needing to wait for relatively longer period such as 3-5 days as required by conventional testing equipment. Thus the present disclosure not only saves time and efforts of testing but also can be very effective in prescribing a correct medication by physicians/doctors based on the availability of accurate and instantaneous results as made possible by the system/sensor of the present disclosure.

### Example-5 (Preparation of the E. coli Biosensor Electrodes/Probes and Measurement of Biological Sample)

An electrode for biosensor-based detection system was prepared for detecting E. coli strain by microfabrication technology by printing carbon-based working electrodes on paper. The printed carbon-based working electrodes were functionalized with 10 μL of 0.5 mg/ml streptavidin at room temperature for 10 minutes. The 10 μL of E. coli specific targeted {(16s RNA gene targeted for E. coli infection (UTI, URI etc. detection/rfb-E gene to target EHEC-haemorrhagic infection/CTX-M gene targeted ESBL producing E. coli for antibiotic resistance)} capture probe was immobilized on the screen printed electrode by at room temperature for 30 minutes, after which the probe was then washed with phosphate buffer saline (having 19 ml of 1M Na₂HPO₄ and 81 ml of 1M K₂HPO₄) having pH of 7.4. The oligonucleotide sequence for detection of rfb-E gene of E. coli de used was as follows: Capture probe SEQ ID NO. 3 and Detector probe SEQ ID NO. 4.

A biological sample containing human urine (or blood sample) was taken from an acute leukaemia patient. The urine sample was used in undiluted form whereas a blood sample was diluted with water in 1:1 ratio before usage. The sample was centrifuged at 14000 rpm for 15 minutes at room temperature to obtain a sedimented portion/pellet of plasmid DNA which was used for further analysis whereas the supernatant was discarded. Further, a 350 μL cocktail mixture was prepared for adding to the pellet obtained from the urine sample for carrying out cell lysis of the gram negative bacterium to be detected. The cocktail mixture included 1 molar sodium hydroxide solution, 10% Tween+20 mM Tris (hydroxymethyl) aminomethane hydrochloride (Tris HCl), 1 mM EDTA, Lysozyme (10 mg/ml) in 10 mM tris HCL, wherein the volume ratio of all the five ingredients was 1:1:1:0.5:0.25. Upon adding the cocktail mixture to the pellet obtained by the centrifugation of the urine sample, the urine sample released target nucleic acid sequence after cell lysis. The resultant mixture was centrifuged at 14000 rpm for 15 minutes and the resultant supernatant solution was taken. A complex based on hybridization between nucleic acid sequence and detector probe was obtained by adding a species specific detector probe to the supernatant solution inside cold box at 65° C. incubator for 10 minutes to form a detector probe-target nucleic acid complex. The detector probe was designed and prepared by tagging a single-stranded oligonucleotide with fluorescein. After that, second hybridization was allowed between the detector probe-target nucleic acid complex with the capture probe-inside a cold box (having temperature of 4° C.) at 65° C. incubator for 15 minutes, forming a three dimensional structure of the target nucleic acid sequence with the capture probe and the detector probe. This was followed by addition of Anti-Fluorescein-POD, Fab fragments from sheep was done for 10 minutes at room temperature and the oxidation-reduction response at the sensor electrodes was measured adding TMB substrate using potentiostat.

### Example 6-E. coli Sensor to Detect Urinary Tract Infection (UTI) and Bloody Diarrhoea in Acute Leukaemia Patient

The bacterial cultures were grown in Luria broth over night at 37° C. at 180 rpm in an incubator cum shaker as per standard guideline. The cells were isolated in log phase where OD between (0.5-1) was used at 600 nm, and further spiked individually with blood and urine sample wherein the urine sample was used in undiluted form whereas blood sample was diluted with water in 1:1. The sensor was standardized for E. coli culture using standard E. coli bacterial isolates and later implemented for detection in the blood and urine spiked with E. coli strain up to 10¹ to 10¹⁰ CFU.

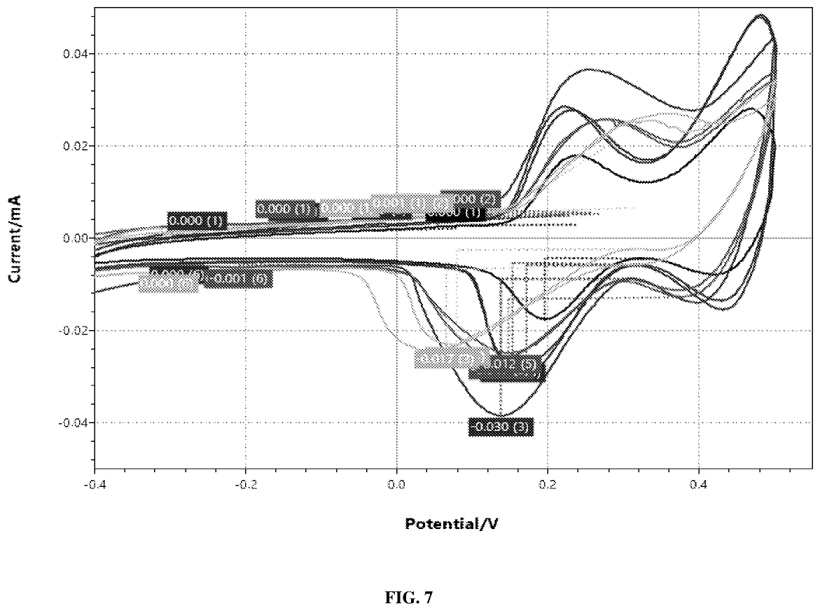

E. coli infection sensor was developed by using MTCC 4296 (UTI pathogenic E. coli strain as positive control for UTI infection. The cyclic voltammetry data for all the dilutions are as provided in Table 8 and in FIG. 7.

TABLE 8
E. coli Probe in diluted samples 01 to 09 of
MTCC 4296 (UTI pathogenic E. coli strain

| Diluted | Dilution | Voltage | Current |
|---|---|---|---|
| samples | level | (V) | (in (μA) |
| 01 | 10⁻¹ | 0.18 | −29.81 |
| 02 | 10⁻² | 0.19 | −23 |
| 03 | 10⁻³ | 0.22 | −19.4 |
| 04 | 10⁻⁴ | 0.18 | −19 |
| 05 | 10⁻⁵ | 0.21 | −17.4 |
| 06 | 10⁻⁶ | 0.18 | −15.9 |
| 07 | 10⁻⁷ | 0.21 | −9.7 |
| 08 | 10⁻⁸ | 0.21 | −8.9 |
| 09 | 10⁻⁹ | 0.21 | −4 |

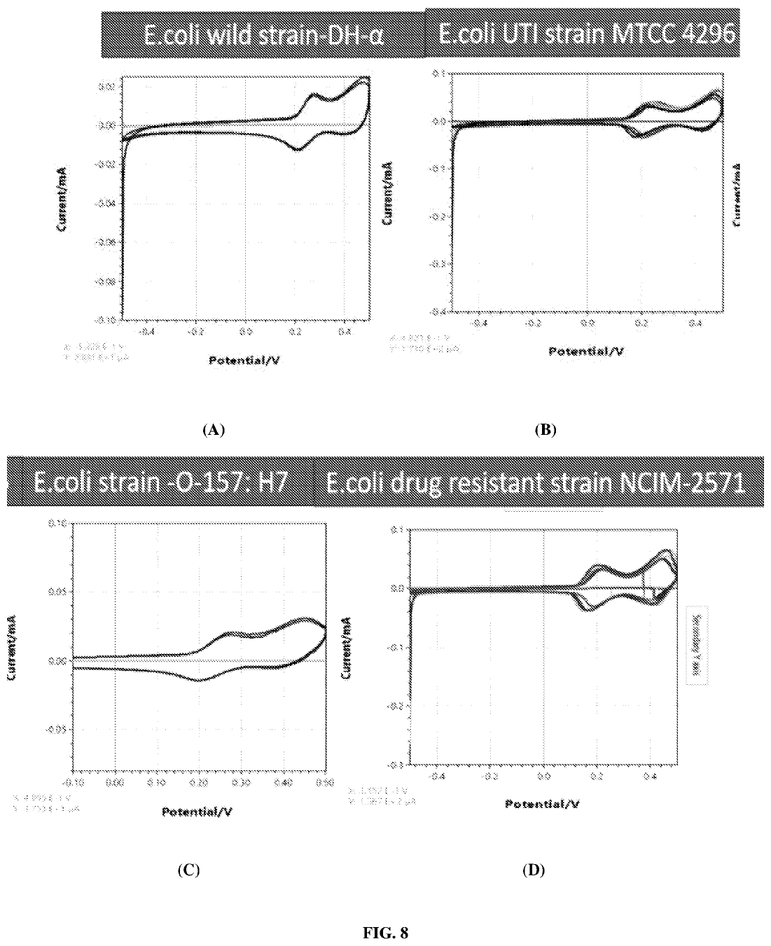

To develop E. coli sensor to detect haemorrhagic diarrhoea, O157-H7 strain was purchased from Microbial Type Culture Collection and Gene Bank, Pune India. The present method was focused on detection of the rfb-E gene to detect the presence of all possible E. coli O157 strains, wherein unique trait of rfb gene to detect all 39 strains (that for O157:H7 and O157:H). The O157 rfb-E PCR assay developed herein provides a selective and rapid technique for confirmation of the O157 serogroup. FIGS. 3A-3D illustrate cyclic voltammetry data that show oxidation reduction cycles of various E. coli probes used in this study, wherein a positive result or presence of a strain of E. coli was known by presence of highest reduction current peak. As illustrated in FIGS. 8A and 8B, 16s RNA based probe shows positivity towards UTI causing E. coli strain MTCC-4296 respectively. Further, as shown in FIG. 8C, rfb gene based probe shows positivity in pathogenic EHEC strain of E. coli O157:H7 and as indicated from FIG. 8D illustrates CTX-M-1 based probe shows positivity in drug resistant strain of E. coli NCIM-2571. This data is further illustrated in a tabular form as given in Table 9 below.

TABLE 9
16S RNA probes in different E. coli strains i.e. DH-5^{α} (wild strain), MTCC-
4296, rfb probe in O-157-H7 and CTX-M probe in ESBL producing E. coli strain NCIM-2571

| 16s RNA in E. Coli | | 16s RNA in E. coli UTI | | Rfb probe in E. coli | | CTX-M1 probe in drug | |
|---|---|---|---|---|---|---|---|
| (Dh-5α strain) | | strain MTCC-4296 | | O-157-H7 strain | | resistant E. coli NCIM-2571 | |
| Voltage | Current | Voltage | Current | Voltage | Current | Voltage | Current |
| (V) | (μA) | (V) | (μA) | (V) | (μA) | (V) | (μA) |
| 0.19 | −13 | 0.19 | −19.9 | 0.18 | −21.24 | 0.15 | −21 |
| 0.21 | −17 | 0.21 | −21.7 | 0.17 | −20.04 | 0.17 | −23 |

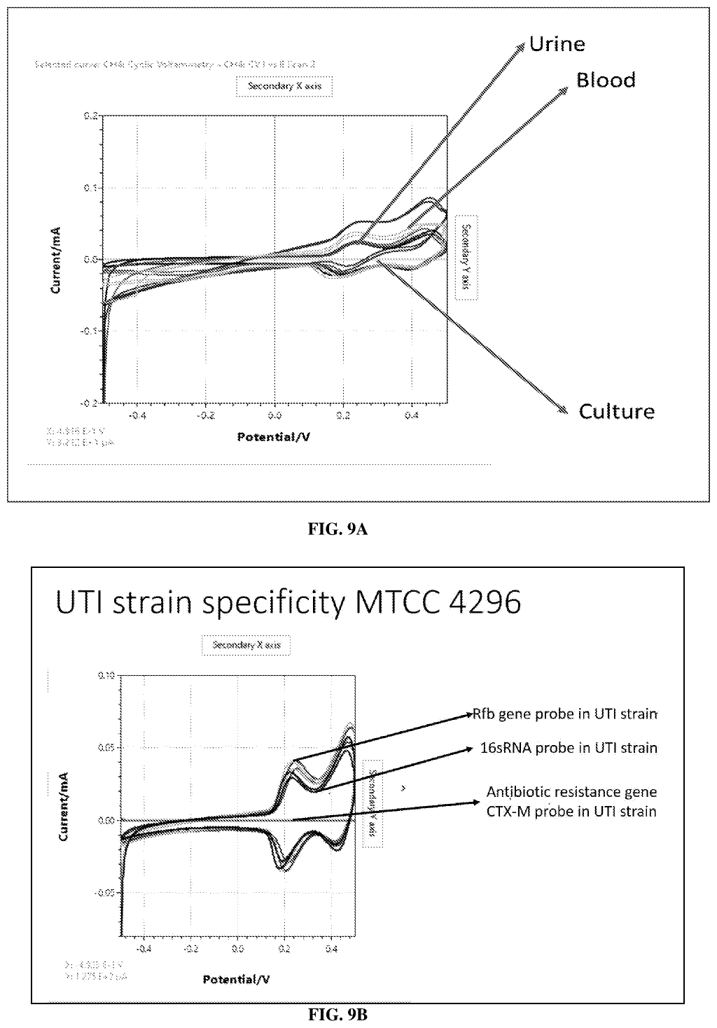

FIG. 9A illustrates cyclic voltammetry data that show oxidation reduction cycles depicting pattern of positivity of 16s RNA probes in term of reduction current in culture of UTI strain and blood and urine. This data is further provided in Table 10 that depicts the Voltage range and highest reduction current of 16s RNA probes in, culture of UTI strain and blood and urine.

TABLE 10
16S RNA probes in UTI Culture, Blood and Urine

| | | |
|---|---|---|
| Culture | Blood | Urine |

| Voltage | Current | Voltage | Current | Voltage | Current |
|---|---|---|---|---|---|
| (V) | (μA) | (V) | (μA) | (V) | (μA) |
| 0.19 | −19.9 | 0.20 | −19.9 | 0.17 | −14.2 |
| 0.21 | −21.7 | 0.21 | −16.12 | 0.19 | −17.23 |

FIG. 9B illustrates cyclic voltammetry data that show oxidation reduction cycles of an infection biosensor having specificity of infection probes corresponding to rfb gene and 16sRNA. It was observed in FIG. 4B that the cyclic voltammetry pattern shows positivity in term of highest reduction current in UTI strain of E. coli whereas CTX-M-1 probe gives negative result in form of blank line.

Thus, in summary two independent E-coli sensors were developed.

i. E. coli Infection Sensor
(A) E. coli (UTI) sensor: UTI sensor targets 16s-RNA gene and gave positive result in biological sample, which predicts UTI infection in acute leukemia as well as patients with other diseases (as shown in FIGS. 8A, 8B, 9A and 9B)
(B) E. coli (haemorrhagic diarrhoea) sensor: E-coli sensor can target rfb gene to detect bloody diarrhoea, which can predict presence of O157 antigen in biological sample and thus can predict the hemorrhagic diarrhoea in acute leukemia patients as well as patients with other diseases (as shown in FIG. 8C).

Hence developing infection sensor could be a helpful tool for physician, as the sensor indicating positive for 16s RNA results would suggest UTI infection whereas rfb gene positivity in biological sample would enable prediction of haemorrhagic diarrhoea in acute leukemia as well as other diseases. In general, the sensor of the present infection can enable detection of species-specific infection caused by E. coli.

### Example 7-E. coli Drug Resistant Sensor

Extended Spectrum β-Lactamases (ESBLs) can hydrolyse monobactams (such as aztreonam), most third-generation cephalosporins (such as cefotaxime, ceftriaxone, and ceftazidime) and, in some cases, even fourth-generation cephalosporins (such as cefepime and cefpirome), hence emerged as the most prominent ESBLs worldwide.

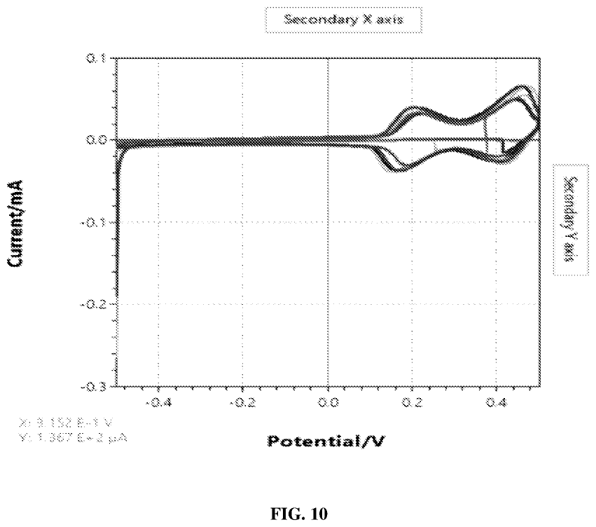

A drug resistant sensor of ESBL (extended-spectrum β-lactamase) producing E. coli was developed using (Cephalosporinase)-CTX-M1 gene specific probe, which target several class of antibiotics. ESBL producing E. coli drug resistant strain NCIM-2571 strain was purchased from NCL (National chemical Laboratory) Pune, India and it was grown in incubator overnight at 37° C. in presence of antibiotics i.e. Cefotaxime 10 μg/ml. Rifampicin 50 μg/ml, Ciprofloxacin lug/ml, Trimethoprim 0.5 μg/ml, ceftazidime and aztreonam as per standard guideline. There is horizontal transfer of blaCTX-M genes, mediated by plasmids and/or mobile elements, contributes to the dissemination of CTX-M enzymes to our community and hospital environments. ESBL production was confirmed using cefotaxime (CTX), ceftazidime (CAZ), and cefepime (FEP) alone and in combination with clavulanic acid (CLA) as per clinical and laboratory standards institute (CLSI) guidelines. The oligonucleotide sequence was used for CTX-M gene as capture probe was SEQ ID NO. 9 and as detector SEQ ID NO. 10. The experimental results as obtained by cyclic voltammetry is as provided in FIG. 10 and in Table 11 below.

TABLE 11
CTX-M1 gene specific probes results in ESBL producing
E. coli drug resistant strain NCIM-2571 strain culture

| Voltage (V) | Current(μA) |
|---|---|
| 0.16 | −23 |
| 0.17 | −26 |
| 0.18 | −20 |
| 0.16 | −28 |
| 0.15 | −28 |
| Blank 0.18 | 0.002 |

ESBL is an enzyme having an ability to hydrolyse the β-lactam ring of broad-spectrum β-lactams such as oxyimino-cephalosporins including cefotaxime, ceftriaxone, and ceftazidime (third generation cephalosporins). ESBL species contain transferrable plasmid containing antimicrobial resistance gene. CTX-M1-gene is also plasmid mediating antimicrobial resistance gene. As indicated in the table 11 and FIG. 10, positive results for CTX-M gene for ESBL producing E. coli in blood or urine sample of patients enables to check the drug resistance in acute leukemia patients having for several class of antibiotics i.e. Cefotaxime, Rifampicin, Ciprofloxacin, Trimethoprim. In short, the multi-drug resistant (MDR) bacteria can be easily detected with CTX-M1 gene specific sensor-based test and hence a physician, who uses the sensor/system of the present disclosure can immediately start treatment within a short duration such as 2 hours after receiving the sample without waiting for duration of 3-5 days, as usually required in conventional testing techniques like PCR and culture.

### Specificity of Sensor to E-Coli:

The specificity of the sensor/probe to E. Coli was also evaluated as provided in Table 12, which shows the specificity of 16 RNA probe positivity various in E. coli strain. As observed in Table 2, except E-coli, other bacterial species such as Klebsiella Pneumonia and Pseudomonas Aeruginosa and drug resistant E. coli display no prominent peaks at the specific voltages, whereas E. coli strain showed positivity in terms of highest reduction current. This indicates the precision and selectively/specificity of the E. Coli.

TABLE 12
Specificity analysis

| | | | | | | | | | | CTX-M probe in | |
|---|---|---|---|---|---|---|---|---|---|---|---|
| | | | | | | E. coli-(MTCC- | | E. Coli -O-157 | | ESBL producing | |
| Klebsiella | | Pseudomonas | | E. Coli | | 4296, Serotype | | Gastric | | E. coli strain | |
| Pneumonia | | Aeruginosa | | DH-α | | 06:K2:H1) | | infection) | | NCIM-2571 | |
| V | μA | V | μA | V | μA | V | μA | V | μA | V | μA |
| 0.13 | −3 | 0.21 | −0.003 | 0.18 | −23 | −0.17 | −24 | 0.18 | −21.24 | 0.22 | 0.08 |
| 0.13 | −7 | 0.2 | −0.001 | 0.17 | −20 | 0.19 | −20 | 0.17 | −20.04 | 0.01 | 0.02 |

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Present Disclosure

The present disclosure provides a biosensor device that can identify infection causing and antibiotic resistant bacterial strain simultaneously in a given sample with >98% sensitivity.

The present disclosure provides a biosensor device capable of DNA-based detection of infection and antibiotic resistance in shorter duration for example less than 2 hours.

The present disclosure provides a device that can detect infection causing and antibiotic resistant bacterial strain with high sensitivity, specificity and in cost effective manner.

The present disclosure provides a kit comprising cassette structure for holding multiple screen printed electrodes for wide range application

The present disclosure provides a biosensor device with a simpler set-up can identification of infection causing and antibiotic resistant bacterial strain in bulk samples detection of infection, or antibiotic resistance, or both.

The present disclosure provides a biosensor device that can provide the result in qualitative and quantitative terms.

The present disclosure provides a biosensor device and kit that can be used as a point of care device for fast detection of infection and/or antibiotic resistance to enable an immediate and appropriate treatment regimen.

The present disclosure provides a biosensor device a kit that can be used by a physician to identify any infection where patient is suffering only from that particular infection or infection in a patient suffering from other diseases.

The present disclosure provides a biosensor device and kit that can be used by a physician as a monitoring tool to treat infection and antibiotic resistance.

1. A portable bio-sensor device for identifying a microorganism in a biological sample for DNA-based detection, the device comprising:
a housing for enclosing one or more components of the device; and
a sensor cabinet provided within the housing, wherein the sensor cabinet comprises a receptacle portion;
wherein the device comprises a potentiometer arrangement that enables measurement of oxidation-reduction process, wherein the potentiometer is configured to apply a pre-defined potential, measure a current output and estimate an amount of cells corresponding to the microorganism present within the sample based on the current output for DNA-based detection.

2. The device as claimed in claim 1, wherein the housing is cuboid-shaped, wherein the device comprises a display screen to indicate the generation data, and wherein the device comprises a thermal printer for printing the generated data.

3. A sensor for identifying a microorganism in a biological sample for DNA-based detection, the sensor comprising a working electrode that interact with at least one capture probe and at least one detector probe, wherein the at least one capture probe comprises a first oligonucleotide sequence and the at least one detector probe comprises a second oligonucleotide sequence, wherein each of the first oligonucleotide sequence and the second oligonucleotide sequence are single-stranded oligonucleotides that are complementary to a nucleic acid sequence hosted by the microorganism, wherein the at least one capture probe and the at least one detector probe upon coming in contact with the nucleotide sequence hosted by the microorganism affords formation of a three-dimensional complex through independent hybridization.

4. The sensor as claimed in claim 3, wherein the formation of the three-dimensional complex is detected by the one or more working electrodes by an oxidation-reduction process that enables generation of an electrical signal for identifying the strain of the microorganism in the sample in the detection of at least one of the infection and the antimicrobial resistance, wherein the sensor comprises a reference electrode and a counter electrode coupled to the working electrode for measurement of the electrical signal in form of dataset including measurement of voltage and current associated with the oxidation-reduction process.

5. The sensor as claimed in claim 3, wherein the at least one capture probe is immobilized on the working electrode by a carrier molecule including protein,
wherein the protein is selected from streptavidin and avidin, wherein the at least one capture probe defines a single-stranded oligonucleotide tagged with a conjugating agent at any of 5′ end or at 3′ end of the oligonucleotide, the conjugating agent being capable of being conjugated with the protein, wherein the conjugating agent is biotin,
wherein the at least one detector probe defines a single-stranded oligonucleotide tagged with a conjugating agent at any of 5′ end or at 3′ end of the oligonucleotide, the conjugating agent being capable of being conjugated with the protein, wherein the conjugating agent is fluorescein.

6. The sensor as claimed in claim 3, wherein the detection of the formation of the three-dimensional complex is done by using one or more reagents selected from any or a combination of anti-fluorescein monoclonal Fab fragment, horseradish peroxidase, a buffer, reagents for cell lysis and detection reagents,
wherein the formation of the three-dimensional complex is carried out inside an acrylamide cassette, wherein the sensor is placed inside the cassette to enable hybridization between the probes and the target nucleic acid sequence on the surface of the sensor.

7. A system for identifying a microorganism in a biological sample for DNA-based detection, the system comprising:
a device as claimed in claim 1; and
a sensor for identifying a microorganism in a biological sample for DNA-based detection, the sensor comprising a working electrode that interact with at least one capture probe and at least one detector probe, wherein the at least one capture probe comprises a first oligonucleotide sequence and the at least one detector probe comprises a second oligonucleotide sequence, wherein each of the first oligonucleotide sequence and the second oligonucleotide sequence are single-stranded oligonucleotides that are complementary to a nucleic acid sequence hosted by the microorganism, wherein the at least one capture probe and the at least one detector probe upon coming in contact with the nucleotide sequence hosted by the microorganism affords formation of a three-dimensional complex through independent hybridization.

8. The system as claimed in claim 7, wherein the system detects the microorganism that is a microbial strain selected from wild type strain, a pathogenic strain, an antibiotic resistant bacterial strain, a multidrug resistant bacterial strain, an extreme drug-resistant strain, and pan drug-resistant strain, wherein the detection of the sample for identifying the microorganism is done in a time duration in the range of 1 minute to 120 minutes, and wherein the biological fluid is selected from blood, urine and other biological fluids of a body.

9. The system as claimed in claim 7, wherein the system enables detection of antimicrobial resistance caused by a gram negative bacterium selected from a strain of E. coli, Klebsiella pneumoniae and Pseudomonas aeruginosa.

10. A set of hybridization probes for hybridizing with a nucleic acid sequence of a microorganism in a sample for DNA-based detection, wherein the set of hybridization probes comprise a capture probe having a first oligonucleotide sequence and a detector probe comprising a second oligonucleotide sequence.

11. The probes as claimed in claim 10, wherein the first oligonucleotide sequence is selected from SEQ ID No. 1, SEQ ID No. 3, SEQ ID No. 5, SEQ ID No. 7, SEQ ID No. 9 and SEQ ID No. 11.

12. The probes as claimed in claim 10, wherein the second oligonucleotide sequence is selected from SEQ ID No. 2, SEQ ID No. 4, SEQ ID No. 6, SEQ ID No. 8, SEQ ID No. 10, SEQ ID No. 12 and SEQ ID No. 14.

13. The probes as claimed in claim 10, the set of hybridization probes are capable of individually hybridizing to any of the target nucleic acid sequence of the gene selected from CTX-M, KPC gene, and NDM-1 gene of Klebsiella pneumoniae strains to identify the strain resistant to antibiotics selected from carbapenem, amino glycosidase and colistin in detection of any or a combination of multi-drug resistance, extensively drug-resistance and pan drug-resistance, and wherein the set of hybridization probes are capable of hybridizing to the target nucleic acid sequence of 16s RNA gene of Klebsiella pneumoniae to identify a virulent strain and to detect the infection,
wherein the set of hybridization probes are capable of individually hybridizing the target nucleic acid sequence of the gene selected from CTX-M, KPC, and NDM-1 of Pseudomonas aeruginosa, to identify the strain resistant to antibiotics selected from carbapenem, amino glycosidase and colistin in detection of any or a combination of multi-drug resistance, extensively drug-resistance and pan drug-resistance.

14. The probes as claimed in claim 10, wherein the set of hybridization probes are capable of individually hybridizing to 16S rRNA of E. coli, Klebsiella pneumoniae and Pseudomonas aeruginosa and the probes are used as marker to identify the pathogenic strain and discriminating from the pathogenic and drug-resistant strains of Pseudomonas aeruginosa,
wherein the set of hybridization probes are capable of individually hybridizing to any of the target nucleic acid sequence of the gene selected from CTX-M, NDM-1, and KPC in extended spectrum beta-lactamase (ESBL) producing E. coli resistant to antibiotics selected from cephalosporins belonging to one to four classes, fluoroquinolones, and monobactam in detection of any or a combination of multi-drug resistance.

15. A kit for identifying a microorganism in a sample for DNA-based detection of an infection and antimicrobial resistance, the kit comprising,
a cocktail mixture capable of lysing the gram negative bacterium and releasing nucleic acid in a biological sample in a time period in the range of 20 to 40 minutes, wherein the cocktail mixture includes a combination of 1M sodium hydroxide solution, 10% Tween, 20 mM Tris (hydroxymethyl) aminomethane hydrochloride (Tris HCl), 1 mM EDTA, Lysozyme (10 mg/ml) in 10 mM tris HCL in a volume ratio of 1:1:1:0.5:0.25;
a set of hybridization probes comprising one or more capture probes and one or more detector probes complementary to and capable of hybridizing to a target nucleic acid sequences of a gene for identification of infection causing and drug resistant strain(s) of the microorganism; and
one or more reagents selected from any or a combination of streptavidin, biotin, anti-fluorescein monoclonal Fab fragment, horseradish peroxidase, a buffer, reagents for cell lysis and detection reagents.


SEQUENCE LISTING (14 sequences)

SEQ ID NO: 1 - DNA, 24 bp, Artificial Sequence
  Part of the sequence was obtained from E.coli having Gene Bank No. J01859.1
ctgcgggtaacgtcaatgagcaaa

SEQ ID NO: 2 - DNA, 24 bp, Artificial Sequence
  Part of the sequence was obtained from E.coli having Gene Bank No. J01859.1
ggtattaactttactcccttcctc

SEQ ID NO: 3 - DNA, 25 bp, Artificial Sequence
  Part of the sequence was obtained from Klebsiella Pneumoneae having Gene Bank No. X87276.1
ctattactacaggtgaaggtggaat

SEQ ID NO: 4 - DNA, 25 bp, Artificial Sequence
  Part of the sequence was obtained from Klebsiella Pneumoneae having Gene Bank No. X87276.1
tcacgaatgacaaaacactttatga

SEQ ID NO: 5 - DNA, 24 bp, Artificial Sequence
  Part of the sequence was obtained from Pseudomonas aurogenosa having Gene Bank No. HG934768.1
ccatgaagtcggaatcgctagtaa

SEQ ID NO: 6 - DNA, 26 bp, Artificial Sequence
  Part of the Sequence was obtained from Pseudomonas aurogenosa having Gene Bank No. HG934768.1
tagatcagaatgctacggtgaatacg

SEQ ID NO: 7 - DNA, 25 bp, Artificial Sequence
  Part of the sequence was obtained from E.coli having Gene Bank No. X92506.1
ctgatactgacactgaggtgcgaaa

SEQ ID NO: 8 - DNA, 25 bp, Artificial Sequence
  Part of the sequence was obtained from E.coli having Gene Bank No. X92506.1
cgtggggagcaaacaggattagata

SEQ ID NO: 9 - DNA, 24 bp, Artificial Sequence
  Part of the sequence was obtained from Pseudomonas aeruginosa having Gene Bank No. HF546976.1
ggcattgattaacacagcagataa

SEQ ID NO: 10 - DNA, 25 bp, Artificial Sequence
  Part of the sequence was obtained from Pseudomonas aeruginosa having Gene Bank No. HF546976.1
caaatactttatcgtgctgatgagc

SEQ ID NO: 11 - DNA, 25 bp, Artificial Sequence
  Part of the sequence was obtained from Klebsiella Pneumoneae having Gene Bank No. AOGQ01000049.1
gtttaatgttggaggctaagtgata

SEQ ID NO: 12 - DNA, 26 bp, Artificial Sequence
  Part of the sequence was obtained from Klebsiella Pneumoneae having Gene Bank No. AOGQ01000049.1
acagtaaggacgcatacaataataag

SEQ ID NO: 13 - DNA, 25 bp, Artificial Sequence
  Part of the sequence is universal having Gene Bank No. HV787867.1
atgtcactgaatactcgtcctagaa

SEQ ID NO: 14 - DNA, 25 bp, Artificial Sequence
  Part of the sequence is universal having Gene Bank No. HV787867.1
cgttagattggcttacaccattaga